(19) United States
(12) Patent Application Publication
(10) Pub. No.: US 2025/0114612 A1
(43) Pub. Date: Apr. 10, 2025

(54) ASSISTED PROGRAMMING SYSTEM FOR NEURAL STIMULATION THERAPY

(71) Applicant: Saluda Medical Pty Ltd, Macquarie Park, New South Wales (AU)

(72) Inventors: Matthew Marlon Williams, Macquarie Park (AU); Daniel John Parker, Macquarie Park (AU); Samuel Nicholas Gilbert, Macquarie Park (AU); Dean Michael Karantonis, Macquarie Park (AU); Epalawattege Sudam Nimantha Dias, Macquarie Park (AU)

(21) Appl. No.: 18/722,972

(22) PCT Filed: Dec. 22, 2022
(86) PCT No.: PCT/AU2022/051556

§ 371 Date: Jun. 21, 2024

(30) Foreign Application Priority Data
Dec. 23, 2021 (AU) 2021904237

(51) Int. Cl. A61N 1/36; A61N 1/372

(52) U.S. Cl. CPC A61N 1/3615; A61N 1/36139; A61N 1/37247

(57) ABSTRACT

Disclosed is an automated programming system for a neuromodulation device that is configured to assist a clinician to efficiently program the neuromodulation device for a particular patient. In particular, the assisted programming system comprises a stimulation user interface control that is configured to cause the intensity of the neural stimulus to ramp up with time as long as the patient continues to interact with the control. The value of stimulus intensity at which the patient ceases to interact with the control is recorded as a significant perceptual marker upon which subsequent steps in the assisted programming workflow are based. This user interface design takes advantage of the human withdrawal reflex, whereby the patient is likely to instinctively release the button upon receiving uncomfortable stimulation. The design therefore minimises the training burden placed on the patient in using the assisted programming system.

### TECHNICAL FIELD

The present invention relates to neural stimulation therapy and in particular to methods and systems for programming a neural stimulation therapy system to suit the needs of a particular patient.

### BACKGROUND OF THE INVENTION

There are a range of situations in which it is desirable to apply neural stimuli in order to alter neural function, a process known as neuromodulation. For example, neuromodulation is used to treat a variety of disorders including chronic neuropathic pain, Parkinson's disease, and migraine. A neuromodulation system applies an electrical pulse (stimulus) to neural tissue (fibres, or neurons) in order to generate a therapeutic effect. In general, the electrical stimulus generated by a neuromodulation system evokes a neural response known as an action potential in a neural fibre which then has either an inhibitory or excitatory effect. Inhibitory effects can be used to modulate an undesired process such as the transmission of pain, or excitatory effects may be used to cause a desired effect such as the contraction of a muscle.

When used to relieve neuropathic pain originating in the trunk and limbs, the electrical pulse is applied to the dorsal column (DC) of the spinal cord, a procedure referred to as spinal cord stimulation (SCS). Such a system typically comprises an implanted electrical pulse generator, and a power source such as a battery that may be transcutaneously rechargeable by wireless means, such as inductive transfer. An electrode array is connected to the pulse generator, and is implanted adjacent the target neural fibre(s) in the spinal cord, typically in the dorsal epidural space above the dorsal column. An electrical pulse of sufficient intensity applied to the target neural fibres by a stimulus electrode causes the depolarisation of neurons in the fibres, which in turn generates an action potential in the fibres. Action potentials propagate along the fibres in orthodromic (towards the head, or rostral) and antidromic (towards the cauda, or caudal) directions. The fibres being stimulated in this way inhibit the transmission of pain from a region of the body innervated by the target neural fibres (the dermatome) to the brain. To sustain the pain relief effects, stimuli are applied repeatedly, for example at a frequency in the range of 30 Hz-100 Hz.

For effective and comfortable neuromodulation, it is necessary to maintain stimulus intensity above a recruitment threshold. Stimuli below the recruitment threshold will fail to recruit sufficient neurons to generate action potentials with a therapeutic effect. In almost all neuromodulation applications, response from a single class of fibre is desired, but the stimulus waveforms employed can evoke action potentials in other classes of fibres which cause unwanted side effects. In pain relief, is therefore necessary to apply stimuli with intensity below a discomfort threshold, above which uncomfortable or painful percepts arise due to over-recruitment of Aβ fibres. When recruitment is too large, Aβ fibres produce uncomfortable sensations. Stimulation at high intensity may even recruit Aδ fibres, which are sensory nerve fibres associated with acute pain, cold and pressure sensation. It is therefore desirable to maintain stimulus intensity within a therapeutic range between the recruitment threshold and the discomfort threshold.

The task of maintaining appropriate neural recruitment is made more difficult by electrode migration (change in position over time) and/or postural changes of the implant recipient (patient), either of which can significantly alter the neural recruitment arising from a given stimulus, and therefore the therapeutic range. There is room in the epidural space for the electrode array to move, and such array movement from migration or posture change alters the electrode-to-fibre distance and thus the recruitment efficacy of a given stimulus. Moreover, the spinal cord itself can move within the cerebrospinal fluid (CSF) with respect to the dura. During postural changes, the amount of CSF and/or the distance between the spinal cord and the electrode can change significantly. This effect is so large that postural changes alone can cause a previously comfortable and effective stimulus regime to become either ineffectual or painful.

Attempts have been made to address such problems by way of feedback or closed-loop control, such as using the methods set forth in International Patent Publication No. WO 2012/155188 by the present applicant. Feedback control seeks to compensate for relative nerve/electrode movement by controlling the intensity of the delivered stimuli so as to maintain a substantially constant neural recruitment. The intensity of a neural response evoked by a stimulus may be used as a feedback variable representative of the amount of neural recruitment. A signal representative of the neural response may be generated by a measurement electrode in electrical communication with the recruited neural fibres, and processed to obtain the feedback variable. Based on the response intensity, the intensity of the applied stimulus may be adjusted to maintain the response intensity within a therapeutic range.

It is therefore desirable to accurately measure the intensity and other characteristics of a neural response evoked by the stimulus. The action potentials generated by the depolarisation of a large number of fibres by a stimulus sum to form a measurable signal known as an evoked compound action potential (ECAP). Accordingly, an ECAP is the sum of responses from a large number of single fibre action potentials. The ECAP generated from the depolarisation of a group of similar fibres may be measured at a measurement electrode as a positive peak potential, then a negative peak, followed by a second positive peak. This morphology is caused by the region of activation passing the measurement electrode as the action potentials propagate along the individual fibres.

Approaches proposed for obtaining a neural response measurement are described by the present applicant in International Patent Publication No. WO 2012/155183, the content of which is incorporated herein by reference.

However, neural response measurement can be a difficult task as an observed CAP signal component in the measured response will typically have a maximum amplitude in the range of microvolts. In contrast, a stimulus applied to evoke the CAP is typically several volts, and manifests in the measured response as crosstalk of that magnitude. Moreover, stimulus generally results in electrode artefact, which manifests in the measured response as a decaying output of the order of several millivolts after the end of the stimulus. As the CAP signal can be contemporaneous with the stimulus crosstalk and/or the stimulus artefact, CAP measurements present a difficult challenge of measurement amplifier design. For example, to resolve a 10 μV CAP with 1 μV resolution in the presence of stimulus crosstalk of 5 V requires an amplifier with a dynamic range of 134 dB, which is impractical in implantable devices. In practice, many non-ideal aspects of a circuit lead to artefact, and as these aspects mostly result a time-decaying artefact waveform of positive or negative polarity, their identification and elimination can be laborious.

Closed-loop neural stimulation therapy is governed by a number of parameters to which values must be assigned to implement the therapy. The effectiveness of the therapy depends in large measure on the suitability of the assigned parameter values to the patient undergoing the therapy. As patients vary significantly in their physiological characteristics, a “one-size-fits-all” approach to parameter value assignment is likely to result in ineffective therapy for a large proportion of patients. An important preliminary task, once a neuromodulation device has been implanted in a patient, is therefore to assign values to the therapy parameters that maximise the effectiveness of the therapy the device will deliver to that particular patient. This task is known as programming or fitting the device. Programming generally involves applying certain test stimuli via the device, recording responses, and based on the recorded responses, inferring or calculating the most effective parameter values for the patient. The resulting parameter values are then formed into a “program” that may be loaded to the device to govern subsequent therapy. Some of the recorded responses may be neural responses evoked by the test stimuli, which provide an objective source of information that may be analysed along with subjective responses elicited from the patient. In an effective programming system, the more responses that are analysed, the more effective the eventual assigned parameter values should be.

However, programming may be costly and time-consuming if unnecessarily prolonged. There is therefore an incentive to minimise the number of test stimuli to be applied and the amount of information to be recorded and analysed in order to produce the assigned values of the therapy parameters. In particular, the size of the therapy parameter search space is such that testing every possible combination of therapy parameters is impractical.

Moreover, programming workflows are generally conducted by a trained clinician or engineer who mediates between the patient and the programming system by interpreting the patient's subjective verbal responses. However, this mediation may be problematic, particularly when patients lack the capacity to express the sensations they are feeling during the test stimuli. In addition, the subjective responses of the patient, even if clearly expressed, are not always a reliable guide to the device's effect on the patient. This can lead to inefficient programming and, in a worst case, ineffective assigned values for therapy parameters.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In this specification, a statement that an element may be “at least one of” a list of options is to be understood to mean that the element may be any one of the listed options, or may be any combination of two or more of the listed options.

### SUMMARY OF THE INVENTION

Disclosed herein is an automated programming system for a neuromodulation device that is configured to assist a clinician to efficiently program the neuromodulation device for a particular patient. In particular, the assisted programming system comprises a stimulation user interface control that is configured to cause the intensity of the neural stimulus to ramp up with time as long as the patient continues to interact with the control. The value of stimulus intensity at which the patient ceases to interact with the control is recorded as a significant perceptual marker upon which subsequent steps in the assisted programming workflow are based. This user interface design takes advantage of the human withdrawal reflex, whereby the patient is likely to instinctively release the button upon receiving uncomfortable stimulation. The design therefore minimises the training burden placed on the patient in using the assisted programming system.

According to a first aspect of the present technology, there is provided a neurostimulation system comprising:
a neurostimulation device for controllably delivering neural stimuli, the neurostimulation device comprising:
   a plurality of implantable electrodes;
   a stimulus source configured to deliver neural stimuli via selected electrodes of the plurality of implantable electrodes to a neural pathway of a patient; and
   a control unit configured to control the stimulus source to deliver each neural stimulus according to a stimulus intensity parameter; and
an external computing device in communication with the neurostimulation device, the external computing device comprising:
   a display, and
   a processor configured to:
      initialise the stimulus intensity parameter;
      render a stimulation control on the display;
      ramp, on receiving an activation of the stimulation control by a user, a value of the stimulus intensity parameter while instructing the control unit to control the stimulus source to deliver the neural stimuli according to the ramping value of the stimulus intensity parameter; and
      cease, upon receiving a de-activation of the stimulation control, ramping the value of the stimulus intensity parameter.

In some embodiments the processor may be further configured to ramp down, upon receiving the de-activation, the value of the stimulus intensity parameter, while instructing the control unit to control the stimulus source to deliver the neural stimuli according to the down-ramping value of the stimulus intensity parameter.

In some embodiments the processor may be further configured to instruct, upon receiving the de-activation, the stimulus source to cease delivering the neural stimuli.

In some embodiments the processor may be further configured to record, upon receiving the de-activation, the value of the stimulus intensity parameter as a discomfort threshold for the selected electrodes. The processor may be further configured to program, using the recorded value of the stimulus intensity parameter, the neurostimulation device to deliver neural stimulus to the patient.

In some embodiments the stimulation control may be configured to:
become activated upon the user interacting with the stimulation control; and
remain activated as long as the user continues to interact with the stimulation control; and
become de-activated as soon as the user ceases to interact with the stimulation control.

In some embodiments the processor may be configured to animate the stimulation control to indicate elapsed time since the activation of the control.

In some embodiments the processor may be configured to animate the stimulation control to indicate that the stimulus intensity has reached a neural response threshold.

In some embodiments the processor may be further configured to cease ramping the value of the stimulus intensity parameter upon expiry of a first timeout period since receipt of a communication from the neurostimulation device. The processor may be further configured to ramp down, upon expiry of a second timeout period since the expiry of the first timeout period without receiving a communication from the neurostimulation device, the value of the stimulus intensity parameter, while instructing the control unit to control the stimulus source to deliver the neural stimuli according to the down-ramping value of the stimulus intensity parameter.

According to a second aspect of the present technology, there is provided an automated method of controlling a neurostimulation device to deliver neural stimuli using an external computing device in communication with the neurostimulation device, the method comprising:
rendering, by a processor of the external computing device, a stimulation control on a display of the external computing device;
ramping, by the processor, on receiving an activation of the stimulation control by a user, a value of a stimulus intensity parameter, while instructing the neurostimulation device to deliver the neural stimuli according to the ramping value of the stimulus intensity parameter via selected electrodes of a plurality of implanted electrodes; and
ceasing, by the processor, upon receiving a de-activation of the stimulation control, to ramp the value of the stimulus intensity parameter.

Some embodiments may further comprise ramping down, by the processor, upon receiving the de-activation, the value of the stimulus intensity parameter, while instructing the neurostimulation device to deliver the neural stimuli according to the down-ramping value of the stimulus intensity parameter via the selected electrodes.

Some embodiments may further comprise ramping down, by the processor, upon the stimulus intensity parameter satisfying a predetermined condition, the value of the stimulus intensity parameter to zero, while instructing the neurostimulation device to deliver the neural stimuli according to the down-ramping value of the stimulus intensity parameter via the selected electrodes. In some embodiments the predetermined condition may comprise the stimulus intensity parameter reaching a hard ceiling.

In some embodiments the down-ramping value of the stimulus intensity parameter may follow a linear profile with a predetermined rate of decrease. A rate of the linear profile may be chosen such that the stimulus intensity parameter reaches zero after a predetermined interval.

In some embodiments the down-ramping value of the stimulus intensity parameter may follow a threshold ramp parametrised by an ECAP threshold.

In some embodiments the ramp may be linear with time.

In some embodiments a ramp rate of the linear ramp may be predetermined.

In some embodiments the ramp may be a threshold ramp parametrised by an ECAP threshold.

Some embodiments may further comprise instructing, by the processor, upon receiving the de-activation, the neurostimulation device to cease delivering the neural stimuli.

Some embodiments may further comprise recording, upon receiving the de-activation, the value of the stimulus intensity parameter as a discomfort threshold for the selected electrodes of the plurality of implanted electrodes.

Some embodiments may further comprise repeating, by the processor, the ramping and instructing and ceasing for further selected electrodes of the plurality of implanted electrodes.

In some embodiments the stimulus intensity parameter may be an amplitude of a stimulus current pulse.

Some embodiments may further comprise animating the stimulation control to indicate elapsed time since the activation of the control. The animating may indicate that the stimulus intensity has reached a neural response threshold.

Some embodiments may further comprise ceasing ramping the value of the stimulus intensity parameter upon expiry of a first timeout period since receipt of a communication from the neurostimulation device. Some embodiments may further comprise ramping down, upon expiry of a second timeout period since the expiry of the first timeout period without receiving a communication from the neurostimulation device, the value of the stimulus intensity parameter, while instructing, by the processor, the neurostimulation device to deliver the neural stimuli according to the down-ramping value of the stimulus intensity parameter.

References herein to estimation, determination, comparison and the like are to be understood as referring to an automated process carried out on data by a processor operating to execute a predefined procedure suitable to effect the described estimation, determination and/or comparison step(s). The technology disclosed herein may be implemented in hardware (e.g., using digital signal processors, application specific integrated circuits (ASICs) or field programmable gate arrays (FPGAs)), or in software (e.g., using instructions tangibly stored on non-transitory computer-readable media for causing a data processing system to perform the steps described herein), or in a combination of hardware and software. The disclosed technology can also be embodied as computer-readable code on a computer-readable medium. The computer-readable medium can include any data storage device that can store data which can thereafter be read by a computer system. Examples of the computer-readable medium include read-only memory (“ROM”), random-access memory (“RAM”), magnetic tape, optical data storage devices, flash storage devices, or any other suitable storage devices. The computer-readable medium can also be distributed over network-coupled computer systems so that the computer-readable code is stored and/or executed in a distributed fashion.

### BRIEF DESCRIPTION OF THE DRAWINGS

One or more implementations of the invention will now be described with reference to the accompanying drawings, in which:

FIG. 1 schematically illustrates an implanted spinal cord stimulator, according to one implementation of the present technology;

FIG. 2 is a block diagram of the stimulator of FIG. 1;

FIG. 3 is a schematic illustrating interaction of the implanted stimulator of FIG. 1 with a nerve;

FIG. 4a illustrates an idealised activation plot for one posture of a patient undergoing neural stimulation;

FIG. 4b illustrates the variation in the activation plots with changing posture of the patient;

FIG. 5 is a schematic illustrating elements and inputs of a closed-loop neural stimulation system, according to one implementation of the present technology;

FIG. 6 illustrates the typical form of an electrically evoked compound action potential (ECAP) of a healthy subject;

FIG. 7 is a block diagram of a neural stimulation therapy system including the implanted stimulator of FIG. 1 according to one implementation of the present technology;

FIG. 8 is a flow chart representing an assisted programming workflow implemented by the assisted programming application according to one implementation of the present technology.

FIG. 9 illustrates the locations of the recording and reference electrodes in the six candidate measurement electrode configurations according to one implementation of the present technology.

FIG. 10 illustrates a screen of the user interface display during a patient-controlled stimulus ramp stage of the workflow of FIG. 8 according to one implementation of the present technology.

FIG. 11a is a flowchart illustrating a data collection and analysis method carried out by the APM and the device during the patient-controlled stimulus ramp stage of the workflow of FIG. 8 according to one implementation of the present technology.

FIG. 11b is a flowchart illustrating a data collection and analysis method carried out by the APM and the device during the patient-controlled stimulus ramp stage of the workflow of FIG. 8 according to an alternative implementation of the present technology.

FIG. 12 illustrates a screen of the user interface display during a coverage survey stage of the workflow of FIG. 8 according to one implementation of the present technology.

FIG. 13 shows a fitted logistic growth curve model to a set of value pairs of stimulus current amplitude and ECAP amplitude, alongside a piecewise linear model fit to the same value pairs.

FIG. 14 illustrates a threshold ramp according to one implementation of the present technology.

FIG. 15 illustrates a screen of the user interface display during a coverage selection stage of the workflow of FIG. 8 according to one implementation of the present technology.

FIG. 16 illustrates a screen of the user interface display during a measurement optimisation stage of the workflow of FIG. 8 according to one implementation of the present technology.

FIG. 17 contains a flowchart illustrating a data collection and analysis method carried out by the APM and the device during the measurement optimisation stage of the workflow of FIG. 8 according to one implementation of the present technology.

FIGS. 18a to 18f illustrate ramps and down-ramps of stimulus intensity according to one implementation of the present technology.

### DETAILED DESCRIPTION OF THE PRESENT TECHNOLOGY

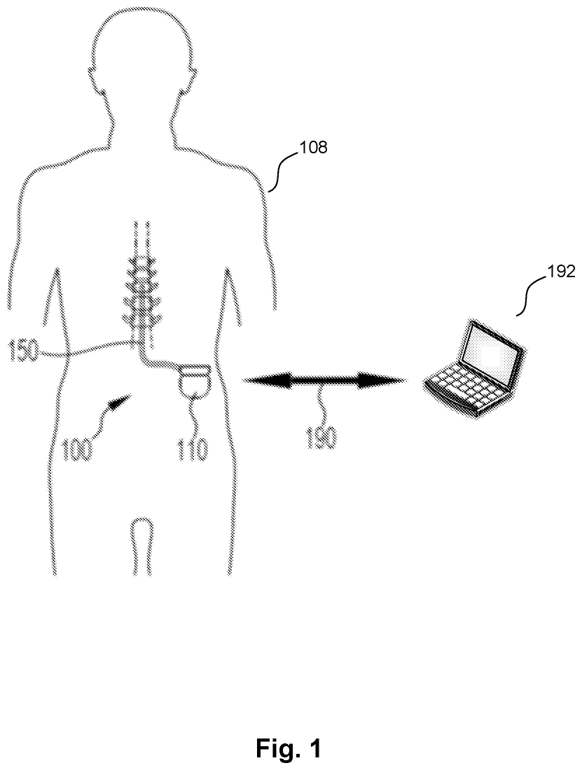

FIG. 1 schematically illustrates an implanted spinal cord stimulator 100 in a patient 108, according to one implementation of the present technology. Stimulator 100 comprises an electronics module 110 implanted at a suitable location. In one implementation, stimulator 100 is implanted in the patient's lower abdominal area or posterior superior gluteal region. In other implementations, the electronics module 110 is implanted in other locations, such as in a flank or sub-clavicularly. Stimulator 100 further comprises an electrode array 150 implanted within the epidural space and connected to the module 110 by a suitable lead. The electrode array 150 may comprise one or more electrodes such as electrode pads on a paddle lead, circular (e.g., ring) electrodes surrounding the body of the lead, conformable electrodes, cuff electrodes, segmented electrodes, or any other type of electrodes capable of forming unipolar, bipolar or multipolar electrode configurations for stimulation and measurement. The electrodes may pierce or affix directly to the tissue itself.

Numerous aspects of the operation of implanted stimulator 100 may be programmable by an external computing device 192, which may be operable by a user such as a clinician or the patient 108. Moreover, implanted stimulator 100 serves a data gathering role, with gathered data being communicated to external device 192 via a transcutaneous communications channel 190. Communications channel 190 may be active on a substantially continuous basis, at periodic intervals, at non-periodic intervals, or upon request from the external device 192. External device 192 may thus provide a clinical interface configured to program the implanted stimulator 100 and recover data stored on the implanted stimulator 100. This configuration is achieved by program instructions collectively referred to as the Clinical Programming Application (CPA) and stored in an instruction memory of the clinical interface.

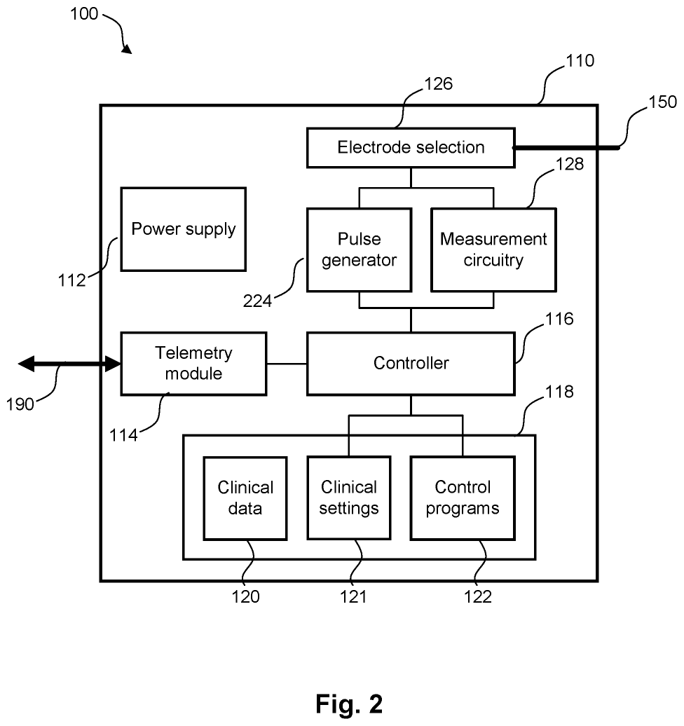

FIG. 2 is a block diagram of the stimulator 100. Electronics module 110 contains a battery 112 and a telemetry module 114. In implementations of the present technology, any suitable type of transcutaneous communications channel 190, such as infrared (IR), radiofrequency (RF), capacitive and/or inductive transfer, may be used by telemetry module 114 to transfer power and/or data to and from the electronics module 110 via communications channel 190. Module controller 116 has an associated memory 118 storing one or more of clinical data 120, clinical settings 121, control programs 122, and the like. Controller 116 controls a pulse generator 124 to generate stimuli, such as in the form of pulses, in accordance with the clinical settings 121 and control programs 122. Electrode selection module 126 switches the generated pulses to the selected electrode(s) of electrode array 150, for delivery of the pulses to the tissue surrounding the selected electrode(s). Measurement circuitry 128, which may comprise an amplifier and/or an analog-to-digital converter (ADC), is configured to process signals comprising neural responses sensed at measurement electrode(s) of the electrode array 150 as selected by electrode selection module 126.

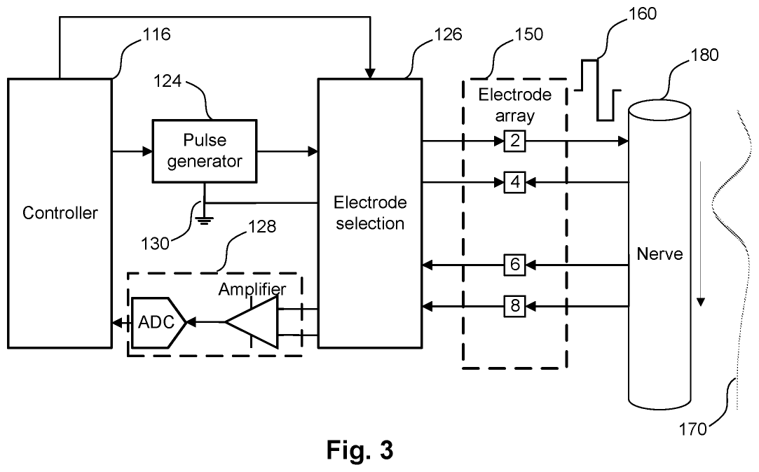

FIG. 3 is a schematic illustrating interaction of the implanted stimulator 100 with a nerve 180 in the patient 108. In the implementation illustrated in FIG. 3 the nerve 180 may be located in the spinal cord, however in alternative implementations the stimulator 100 may be positioned adjacent any desired neural tissue including a peripheral nerve, visceral nerve, parasympathetic nerve or a brain structure. Electrode selection module 126 selects a stimulus electrode 2 of electrode array 150 through which to deliver a pulse from the pulse generator 124 to surrounding tissue including nerve 180. A pulse may comprise one or more phases, e.g. a biphasic stimulus pulse 160 comprises two phases. Electrode selection module 126 also selects a return electrode 4 of the electrode array 150 for stimulus current return in each phase, to maintain a zero net charge transfer. An electrode may act as both a stimulus electrode and a return electrode over a complete multiphasic stimulus pulse. The use of two electrodes in this manner for delivering and returning current in each stimulus phase is referred to as bipolar stimulation. Alternative embodiments may apply other forms of bipolar stimulation, or may use a greater number of stimulus and/or return electrodes, e.g. three electrodes for tripolar stimulation. The set of stimulus electrodes and return electrodes is referred to as the stimulus electrode configuration (SEC). Electrode selection module 126 is illustrated as connecting to a ground 130 of the pulse generator 124 to enable stimulus current return via the return electrode 4. However, other connections for charge recovery may be used in other implementations.

Delivery of an appropriate stimulus from electrodes 2 and 4 to the nerve 180 evokes a neural response 170 comprising an evoked compound action potential (ECAP) which will propagate along the nerve 180 as illustrated at a rate known as the conduction velocity. The ECAP may be evoked for therapeutic purposes, which in the case of a spinal cord stimulator for chronic pain may be to create paraesthesia at a desired location. To this end, the electrodes 2 and 4 are used to deliver stimuli periodically at any therapeutically suitable frequency, for example 30 Hz, although other frequencies may be used including frequencies as high as the kHz range. In alternative implementations, stimuli may be delivered in a non-periodic manner such as in bursts, or sporadically, as appropriate for the patient 108. To program the stimulator 100 to the patient 108, a clinician may cause the stimulator 100 to deliver stimuli of various configurations which seek to produce a sensation that is experienced by the user as paraesthesia. When a stimulus electrode configuration (SEC) is found which evokes paraesthesia in a location and of a size which is congruent with the area of the patient's body affected by pain, the clinician nominates that configuration for ongoing use. The therapy parameters may be loaded into the memory 118 of the stimulator 100 as the clinical settings 121.

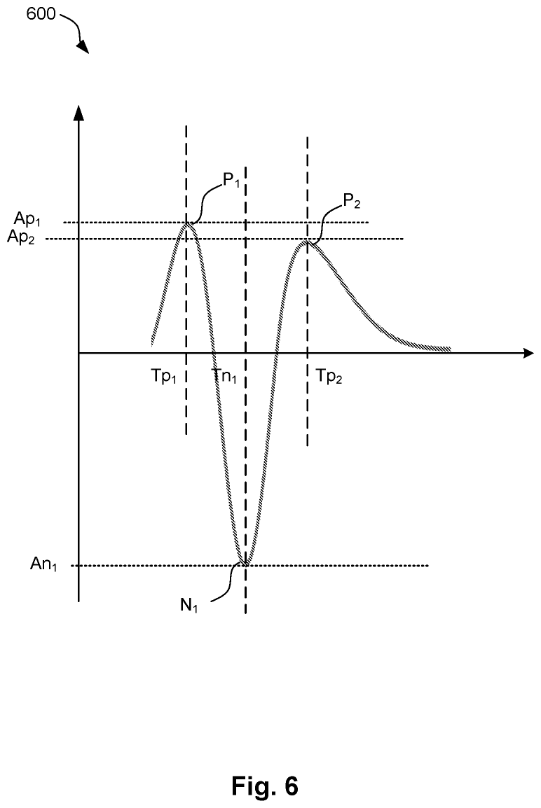

FIG. 6 illustrates the typical form of an ECAP 600 of a healthy subject, as recorded at a single measurement electrode referenced to the system ground 130. The shape and duration of the ECAP 600 shown in FIG. 6 is predictable because it is a result of the ion currents produced by the ensemble of fibres depolarising and generating action potentials (APs) in response to stimulation. The evoked action potentials (EAPs) generated synchronously among a large number of fibres sum to form the ECAP 600. The ECAP 600 generated from the synchronous depolarisation of a group of similar fibres comprises a positive peak P1, then a negative peak N1, followed by a second positive peak P2. This shape is caused by the region of activation passing the measurement electrode as the action potentials propagate along the individual fibres.

The ECAP may be recorded differentially using two measurement electrodes, as illustrated in FIG. 3. Depending on the polarity of recording, a differential ECAP may take an inverse form to that shown in FIG. 6, i.e. a form having two negative peaks N1 and N2, and one positive peak P1. Alternatively, depending on the distance between the two measurement electrodes, a differential ECAP may resemble the time derivative of the ECAP 600, or more generally the difference between the ECAP 600 and a time-delayed copy thereof.

The ECAP 600 may be characterised by any suitable characteristic(s) of which some are indicated in FIG. 6. The amplitude of the positive peak P1 is Ap₁ and occurs at time Tp₁. The amplitude of the positive peak P2 is Ap₂ and occurs at time Tp₂. The amplitude of the negative peak P1 is An₁ and occurs at time Tn₁. The peak-to-peak amplitude is Ap₁+An₁. A recorded ECAP will typically have a maximum peak-to-peak amplitude in the range of microvolts and a duration of 2 to 3 ms.

The stimulator 100 is further configured to measure the intensity of ECAPs 170 propagating along nerve 180, whether such ECAPs are evoked by the stimulus from electrodes 2 and 4, or otherwise evoked. To this end, any electrodes of the array 150 may be selected by the electrode selection module 126 to serve as measurement electrode 6 and measurement reference electrode 8, whereby the electrode selection module 126 selectively connects the chosen electrodes to the inputs of the measurement circuitry 128. Thus, signals sensed by the measurement electrodes 6 and 8 subsequent to the respective stimuli are passed to the measurement circuitry 128, which may comprise a differential amplifier and an analog-to-digital converter (ADC), as illustrated in FIG. 3. The measurement circuitry 128 for example may operate in accordance with the teachings of the above-mentioned International Patent Application Publication No. WO 2012/155183.

Signals sensed by the measurement electrodes 6, 8 and processed by measurement circuitry 128 are further processed by an ECAP detector implemented within controller 116, configured by control programs 122, to obtain information regarding the effect of the applied stimulus upon the nerve 180. In some implementations, the sensed signals are processed by the ECAP detector in a manner which measures and stores one or more characteristics from each evoked neural response or group of evoked neural responses contained in the sensed signal. In one such implementation, the characteristic comprises a peak-to-peak ECAP amplitude in microvolts (μV). For example, the sensed signals may be processed by the ECAP detector to determine the peak-to-peak ECAP amplitude in accordance with the teachings of International Patent Publication No. WO 2015/074121 by the present applicant, the contents of which are incorporated herein by reference. Alternative implementations of the ECAP detector may measure and store an alternative characteristic from the neural response, or may measure and store two or more characteristics from the neural response.

Stimulator 100 applies stimuli over a potentially long period such as days, weeks, or months and during this time may store characteristics of neural responses, stimulation settings, paraesthesia target level, and other operational parameters in memory 118. To effect suitable SCS therapy, stimulator 100 may deliver tens, hundreds or even thousands of stimuli per second, for many hours each day. Each neural response or group of responses generates one or more characteristics such as a measure of the intensity of the neural response. Stimulator 100 thus may produce such data at a rate of tens or hundreds of Hz, or even kHz, and over the course of hours or days this process results in large amounts of clinical data 120 which may be stored in the memory 118. Memory 118 is however necessarily of limited capacity and care is thus required to select compact data forms for storage into the memory 118, to ensure that the memory 118 is not exhausted before such time that the data is expected to be retrieved wirelessly by external device 192, which may occur only once or twice a day, or less.

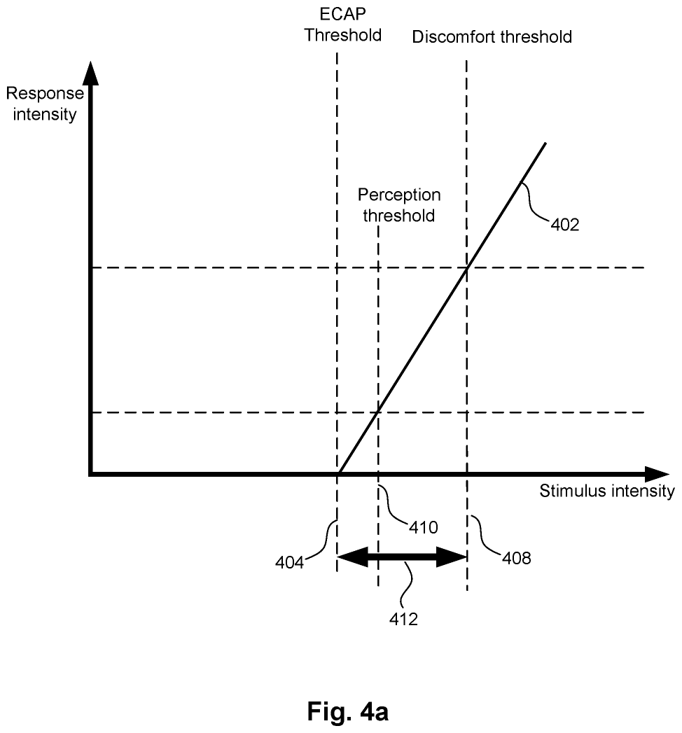

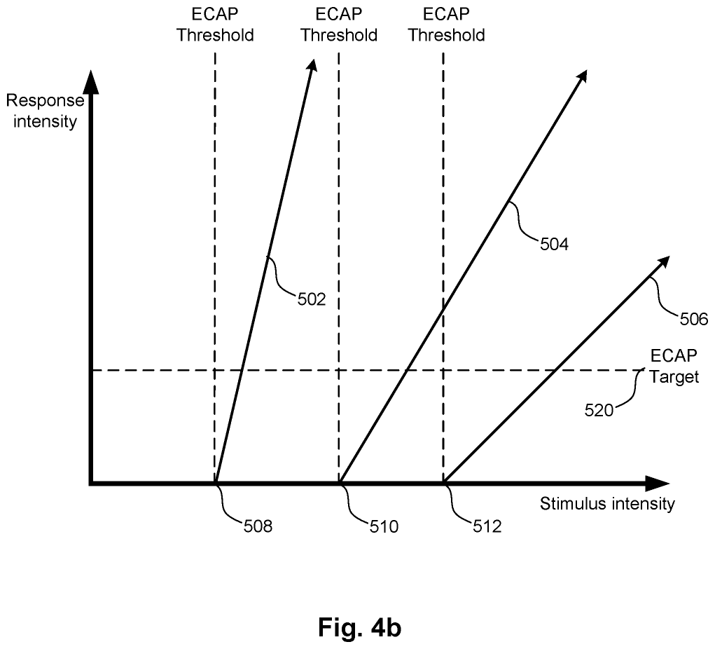

An activation plot, or growth curve, is an approximation to the relationship between stimulus intensity (e.g. an amplitude of the current pulse 160) and intensity of neural response 170 resulting from the stimulus (e.g. an ECAP peak-to-peak amplitude). FIG. 4a illustrates an idealised activation plot 402 for one posture of the patient 108. The activation plot 402 shows a linearly increasing ECAP amplitude for stimulus intensity values above a threshold 404 referred to as the ECAP threshold. The ECAP threshold exists because of the binary nature of fibre recruitment; if the field strength is too low, no fibres will be recruited. However, once the field strength exceeds a threshold, fibres begin to be recruited, and their individual evoked action potentials are independent of the strength of the field. The ECAP threshold 404 therefore reflects the field strength at which significant numbers of fibres begin to be recruited, and the increase in response intensity with stimulus intensity above the ECAP threshold reflects increasing numbers of fibres being recruited. Below the ECAP threshold 404, the ECAP amplitude may be taken to be zero. Above the ECAP threshold 404, the activation plot 402 has a positive, approximately constant slope indicating a linear relationship between stimulus intensity and the ECAP amplitude. Such a relationship may be modelled as:

$$y = \{ \begin{matrix}S ( s - T ) , & s \geq T \\ 0 , & s < T\end{matrix} \qquad ( 1 )$$

where s is the stimulus intensity, y is the ECAP amplitude, Tis the ECAP threshold and S is the slope of the activation plot (referred to herein as the patient sensitivity). The slope S and the ECAP threshold T are the key parameters of the activation plot 402. The ECAP threshold is an example of a physiological threshold.

FIG. 4a also illustrates a discomfort threshold 408, which is a stimulus intensity above which the patient 108 experiences uncomfortable or painful stimulation. FIG. 4 also illustrates a perception threshold 410. The perception threshold 410 is a stimulus intensity that corresponds to an ECAP amplitude that is perceivable by the patient. There are a number of factors which can influence the position of the perception threshold 410, including the posture of the patient. Perception threshold 410 may be a stimulus intensity that is greater than the ECAP threshold 404, as illustrated in FIG. 4a, if patient 108 does not perceive low levels of neural activation. Conversely, the perception threshold 410 may be a stimulus intensity that is less than the ECAP threshold 404, if the patient has a high perception sensitivity to lower levels of neural activation than can be detected in an ECAP, or if the signal to noise ratio of the ECAP is low. The discomfort threshold 408 and perception threshold 410 are examples of perceptual markers for the patient 108.

For effective and comfortable operation of an implantable neuromodulation device such as the stimulator 100, it is desirable to maintain stimulus intensity within a therapeutic range. A stimulus intensity within a therapeutic range 412 is above the ECAP threshold 404 and below the discomfort threshold 408. In principle, it would be straightforward to measure these limits and ensure that stimulus intensity, which may be closely controlled, always falls within the therapeutic range 412. However, the activation plot, and therefore the therapeutic range 412, varies with the posture of the patient 108.

FIG. 4b illustrates the variation in the activation plots with changing posture of the patient. A change in posture of the patient may cause a change in impedance of the electrode-tissue interface or a change in the distance between electrodes and the neurons. While the activation plots for only three postures, 502, 504 and 506, are shown in FIG. 4b, the activation plot for any given posture can lie between or outside the activation plots shown, on a continuously varying basis depending on posture. Consequently, as the patient's posture changes, the ECAP threshold changes, as indicated by the ECAP thresholds 508, 510, and 512 for the respective activation plots 502, 504, and 506. Additionally, as the patient's posture changes, the slope of the activation plot also changes, as indicated by the varying slopes of activation plots 502, 504, and 506. In general, as the distance between the stimulus electrodes and the spinal cord increases, the ECAP threshold increases and the slope of the activation plot decreases. The activation plots 502, 504, and 506 therefore correspond to increasing distance between stimulus electrodes and spinal cord, and decreasing patient sensitivity.

To keep the applied stimulus intensity within the therapeutic range as patient posture varies, in some implementations an implantable neuromodulation device such as the stimulator 100 may adjust the applied stimulus intensity based on a feedback variable that is determined from one or more measured ECAP characteristics. In one implementation, the device may adjust the stimulus intensity to maintain the measured ECAP amplitude at a target response intensity. For example, the device may calculate an error between a target ECAP amplitude and a measured ECAP amplitude, and adjust the applied stimulus intensity to reduce the error as much as possible, such as by adding the scaled error to the current stimulus intensity. A neuromodulation device that operates by adjusting the applied stimulus intensity based on a measured ECAP characteristic is said to be operating in closed-loop mode and will also be referred to as a closed-loop neural stimulus (CLNS) device. By adjusting the applied stimulus intensity to maintain the measured ECAP amplitude at an appropriate target response intensity, such as an ECAP target 520 illustrated in FIG. 4b, a CLNS device will generally keep the stimulus intensity within the therapeutic range as patient posture varies.

A CLNS device comprises a stimulator that takes a stimulus intensity value and converts it into a neural stimulus comprising a sequence of electrical pulses according to a predefined stimulation pattern. The stimulation pattern is parametrised by multiple stimulus parameters including stimulus amplitude, pulse width, number of phases, order of phases, number of stimulus electrode poles (two for bipolar, three for tripolar etc.), and stimulus rate or frequency. At least one of the stimulus parameters, for example the stimulus amplitude, is controlled by the feedback loop.

In an example CLNS system, a user (e.g. the patient or a clinician) sets a target response intensity, and the CLNS device performs proportional-integral-differential (PID) control. In some implementations, the differential contribution is disregarded and the CLNS device uses a first order integrating feedback loop. The stimulator produces stimulus in accordance with a stimulus intensity parameter, which evokes a neural response in the patient. The intensity of an evoked neural response (e.g. an ECAP) is measured by the CLNS device and compared to the target response intensity.

The measured neural response intensity, and its deviation from the target response intensity, is used by the feedback loop to determine possible adjustments to the stimulus intensity parameter to maintain the neural response at the target intensity. If the target intensity is properly chosen, the patient receives consistently comfortable and therapeutic stimulation through posture changes and other perturbations to the stimulus/response behaviour.

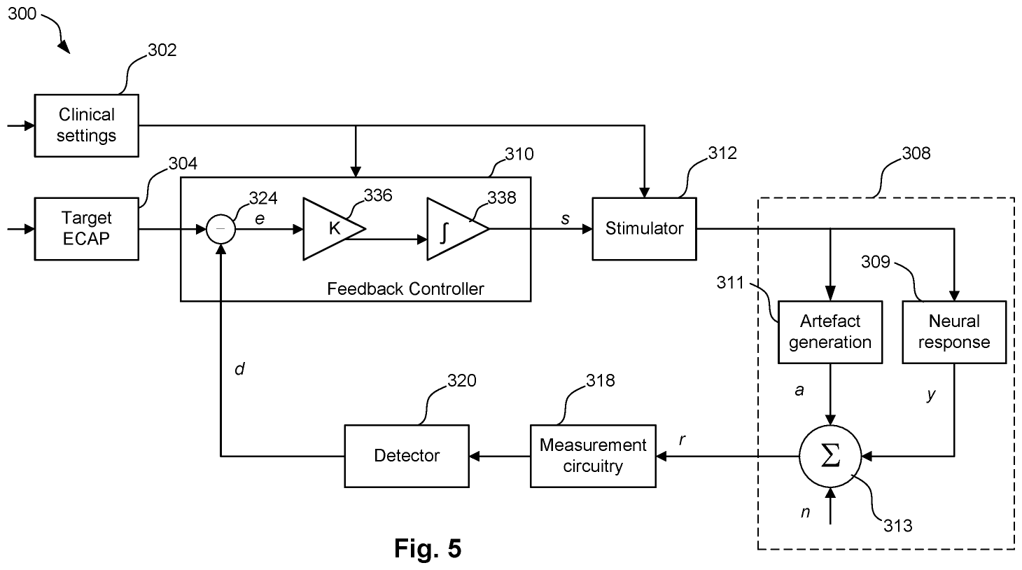

FIG. 5 is a schematic illustrating elements and inputs of a closed-loop neural stimulation system (CLNS) 300, according to one implementation of the present technology. The system 300 comprises a stimulator 312 which converts a stimulus intensity parameter (for example a stimulus current amplitude) s, in accordance with a set of predefined stimulus parameters, to a neural stimulus comprising a sequence of electrical pulses on the stimulus electrodes (not shown in FIG. 5). According to one implementation, the predefined stimulus parameters comprise the number and order of phases, the number of stimulus electrode poles, the pulse width, and the stimulus rate or frequency.

The generated stimulus crosses from the electrodes to the spinal cord, which is represented in FIG. 5 by the dashed box 308. The box 309 represents the evocation of a neural response y by the stimulus as described above. The box 311 represents the evocation of an artefact signal a, which is dependent on stimulus intensity and other stimulus parameters, as well as the electrical environment of the measurement electrode. Various sources of noise n may add to the evoked response y at the summing element 313 before the evoked response is measured, including: electrical noise from external sources such as 50 Hz mains power; electrical disturbances produced by the body such as neural responses evoked not by the device but by other causes such as peripheral sensory input; EEG; EMG; and electrical noise from measurement circuitry 318.

The neural recruitment arising from the stimulus is affected by mechanical changes, including posture changes, walking, breathing, heartbeat and so on. Mechanical changes may cause impedance changes, or changes in the location and orientation of the nerve fibres relative to the electrode array(s). As described above, the intensity of the evoked response provides a measure of the recruitment of the fibres being stimulated. In general, the more intense the stimulus, the more recruitment and the more intense the evoked response. An evoked response typically has a maximum amplitude in the range of microvolts, whereas the voltage resulting from the stimulus applied to evoke the response is typically several volts.

Measurement circuitry 318, which may be identified with measurement circuitry 128, amplifies the sensed signal r (including evoked neural response, artefact, and noise) and samples the amplified sensed signal r to capture a “signal window” comprising a predetermined number of samples of the amplified sensed signal r. The ECAP detector 320 processes the signal window and outputs a measured neural response intensity d. In one implementation, the neural response intensity comprises an ECAP amplitude. The measured response intensity d is input into the feedback controller 310. The feedback controller 310 comprises a comparator 324 that compares the measured response intensity d to a target ECAP amplitude as set by the target ECAP controller 304 and provides an indication of the difference between the measured response intensity d and the target ECAP amplitude. This difference is the error value, e.

The feedback controller 310 calculates an adjusted stimulus intensity parameter, s, with the aim of maintaining a measured response intensity d equal to the target ECAP amplitude. Accordingly, the feedback controller 310 adjusts the stimulus intensity parameter s to minimise the error value, e. In one implementation, the controller 310 utilises a first order integrating function, using a gain element 336 and an integrator 338, in order to provide suitable adjustment to the stimulus intensity parameter s. According to such an implementation, the current stimulus intensity parameter s may be computed by the feedback controller 310 as

$$s = \int Kedt \qquad ( 2 )$$

where K is the gain of the gain element 336 (the controller gain). This relation may also be represented as

δs=Ke

where δs is an adjustment to the current stimulus intensity parameter s.

A target ECAP amplitude is input to the comparator 324 via the target ECAP controller 304. In one embodiment, the target ECAP controller 304 provides an indication of a specific target ECAP amplitude. In another embodiment, the target ECAP controller 304 provides an indication to increase or to decrease the present target ECAP amplitude. The target ECAP controller 304 may comprise an input into the neural stimulus device, via which the patient or clinician can input a target ECAP amplitude, or indication thereof. The target ECAP controller 304 may comprise memory in which the target ECAP amplitude is stored, and from which the target ECAP amplitude is provided to the feedback controller 310.

A clinical settings controller 302 provides therapy parameters to the system, including the gain K for the gain element 336 and the stimulation parameters for the stimulator 312. The clinical settings controller 302 may be configured to adjust the gain K of the gain element 336 to adapt the feedback loop to patient sensitivity. The clinical settings controller 302 may comprise an input into the neural stimulus device, via which the patient or clinician can adjust the therapy parameters. The clinical settings controller 302 may comprise memory in which the therapy parameters are stored, and are provided to components of the system 300.

In some implementations, two clocks (not shown) are used, being a stimulus clock operating at the stimulus frequency (e.g. 60 Hz) and a sample clock for sampling the measured response r (for example, operating at a sampling frequency of 10 kHz). As the ECAP detector 320 is linear, only the stimulus clock affects the dynamics of the CLNS system 300. On the next stimulus clock cycle, the stimulator 312 outputs a stimulus in accordance with the adjusted stimulus intensity s. Accordingly, there is a delay of one stimulus clock cycle before the stimulus intensity is updated in light of the error value e.

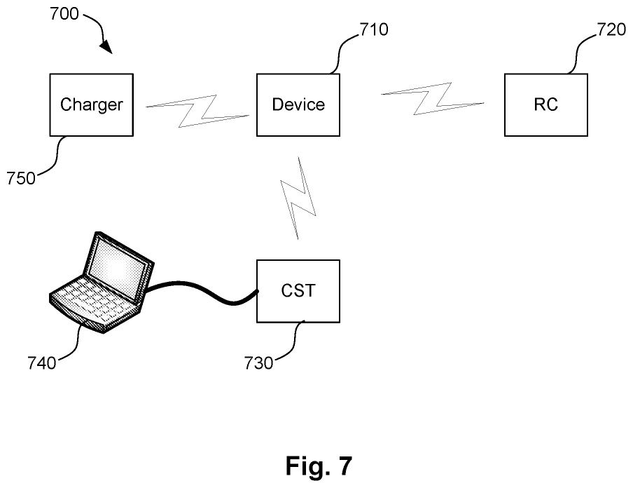

FIG. 7 is a block diagram of a neural stimulation system 700. The neural stimulation system 700 is centred on a neuromodulation device 710. In one example, the neuromodulation device 710 may be implemented as the stimulator 100 of FIG. 1, implanted within a patient (not shown). The neuromodulation device 710 is connected wirelessly to a remote controller (RC) 720. The remote controller 720 is a portable computing device that provides the patient with control of their stimulation in the home environment by allowing control of the functionality of the neuromodulation device 710, including one or more of the following functions: enabling or disabling stimulation; adjustment of stimulus intensity or target response intensity; and selection of a stimulation control program from the control programs stored on the neuromodulation device 710.

The charger 750 is configured to recharge a rechargeable power source of the neuromodulation device 710. The recharging is illustrated as wireless in FIG. 7 but may be wired in alternative implementations.

The neuromodulation device 710 is wirelessly connected to a Clinical System Transceiver (CST) 730. The wireless connection may be implemented as the transcutaneous communications channel 190 of FIG. 1. The CST 730 acts as an intermediary between the neuromodulation device 710 and the Clinical Interface (CI) 740, to which the CST 730 is connected. A wired connection is shown in FIG. 7, but in other implementations, the connection between the CST 730 and the CI 740 is wireless.

The CI 740 may be implemented as the external computing device 192 of FIG. 1. The CI 740 is configured to program the neuromodulation device 710 and recover data stored on the neuromodulation device 710. This configuration is achieved by program instructions collectively referred to as the Clinical Programming Application (CPA) and stored in an instruction memory of the CI 740.

The CPA makes use of a user interface (UI) of the CI 740. The UI may comprise a device for displaying information to the user (e.g. a display) and a device for receiving input from the user, such as a touchscreen, movable pointing device controlling a cursor (mouse), keyboard, joystick, touchpad, trackball etc. In the example of a touchscreen, the input device may be combined with the display. Alternatively, the UI of the CI 740 the input device(s) may be separate from the display.

### The Assisted Programming System

As mentioned above, obtaining patient feedback about their sensations is important during programming of closed-loop neural stimulation therapy, but mediation by trained clinical engineers is expensive and time-consuming. It would therefore be advantageous if patients could program their own implantable device themselves, or with some assistance from a clinician. However, interfaces for current programming systems are non-intuitive and generally unsuitable for direct use by patients because of their technical nature. There is therefore a need for a CPA to be as intuitive for non-technical users as possible while avoiding discomfort to the patient.

Implementations of an Assisted Programming System (APS) according to the present technology are generally configured to meet the needs above. In some implementations, the APS comprises two elements: the Assisted Programming Module (APM), which forms part of the CPA, and the Assisted Programming Firmware (APF), which forms part of the control programs 122 executed by the controller 116 of the electronics module 110. The data obtained from the patient, both subjective and objective, is analysed by the APM to determine the clinical settings for the neural stimulation therapy to be delivered by the stimulator 100. The APF is configured to complement the operation of the APM by responding to commands issued by the APM via the CST 730 to the stimulator 100 to deliver specified stimuli to the patient, and by returning, via the CST 730, measurements of neural responses to the delivered stimuli.

The APS instructs the device 710 to capture and return signal windows to the CI 740 via the CST 730. In such implementations, the device 710 captures the signal windows using the measurement circuit 128 and bypasses the ECAP detector 320, storing the data representing the raw signal windows temporarily in memory 118 before transmitting the data representing the captured signal windows to the APS for analysis.

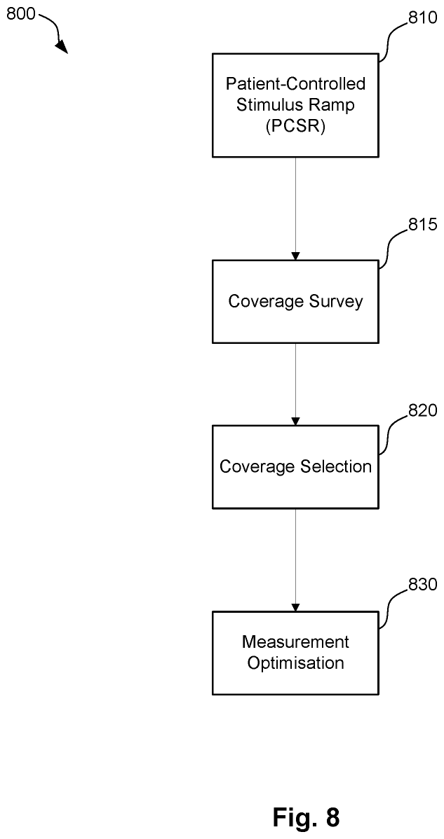

FIG. 8 is a flow chart representing an assisted programming workflow 800 implemented by the APM at a high level, according to one implementation of the present technology. In the assisted programming workflow 800, control of the CI 740 is handed over to a user, for example the patient, who interacts with the APM for the entirety of the workflow. In some implementations, the patient remains in a fixed predetermined posture throughout the workflow. Having direct patient involvement allows for faster feedback because subjective responses to stimulation do not have to be communicated via a clinician. However, the workflow 800 is just one possible implementation of an APM, and it should be noted that there is no formal requirement for any part of the assisted programming system to include direct patient involvement.

The workflow 800 has several stages: a Patient Controlled Stimulus Ramp (PCSR) stage 810, an (optional) Coverage Survey stage 815, a Coverage Selection stage 820, and a Measurement Optimisation (MO) stage 830.

The PCSR stage 810 is configured to deliver stimulus of a gradually increasing intensity and receive subjective input from the patient as to a maximum value of stimulus intensity (“Max” value) for each of one or more candidate stimulus electrode configurations (SECs). The Max value may be identified with the discomfort threshold 408 of FIG. 4a. Meanwhile, the APM is configured to record sensed signals and analyse the recorded data, as well as the patient's Max value for each SEC, to calculate an ECAP Threshold for each SEC. The PCSR stage 810 is described in more detail below.

The Coverage Survey stage 815 is configured to receive input from the patient concerning their sensations in response to stimuli delivered via each candidate SEC at a comfortable stimulus intensity. The comfortable stimulus intensity is predicted for each candidate SEC based on the Max and/or ECAP Threshold values derived in the PCSR stage 810. Based on the patient input, the comfortable stimulus intensity at each SEC may be adjusted. In addition, if stimulus delivered via any candidate SEC feels uncomfortable to the patient in an area of the body, the candidate SEC itself may be adjusted and the PCSR stage 810 is repeated for the adjusted SEC. The Coverage Survey stage 815 is described in more detail below.

The Coverage Selection stage 820 is configured to receive input from the patient to select one or more of the candidate SECs after any adjustments made by the Coverage Survey stage 815. The comfortable stimulus intensity delivered via each candidate SEC is based on the comfortable stimulus intensity derived for that SEC in the PCSR stage 810 and possibly adjusted at the Coverage Survey stage 815. The patient can test different combinations of SECs before selecting which ones to keep. The Coverage Selection stage 820 is described in more detail below.

The Measurement Optimisation (MO) stage 830 is configured to deliver stimulus of a gradually increasing intensity from a primary SEC of the selected SECs, and record sensed signal data at each of multiple measurement electrode configurations (MECs) for the primary SEC. The MO stage 830 is then configured to choose the optimal MEC for the primary SEC based on the response data, calculate physiological characteristics of the patient, and choose optimal therapy parameters for the primary SEC/optimal MEC combination. The selected SECs, including the primary SEC, the optimal MEC, and optimal therapy parameters are referred to as the determined program. The Measurement Optimisation stage 830 is described in more detail below.

Following the workflow 800, if successful, the APS may load the determined program onto the device 710 to govern subsequent neural stimulation therapy. In one implementation, the program comprises clinical settings 121, also referred to as therapy parameters, that are input to the neuromodulation device 710 by, or stored in, the clinical settings controller 302. The patient may subsequently control the device 710 to deliver the therapy according to the determined program using the remote controller 720 as described above. The determined program may also, or alternatively, be loaded into the CPA for validation and modification. Validation and modification of the determined program may also be carried out by the APS itself. If unsuccessful, the device 710 may be manually programmed.

In the workflow 800, the APM may use predetermined values of certain therapy parameters. In one implementation, those parameters and values are:
Stimulus frequency: 40 Hz
Pulse width: 240 microseconds
Inter-phase gap: 200 microseconds
Pulse shape: triphasic, with anodic phase first
Signal window length: 60 samples
Sampling frequency: 16 kHz
Inter-stimulus interval: 5 ms

In one implementation of the workflow 800, four candidate stimulus electrode configurations (SECs) are defined. Each SEC is tripolar, comprising a stimulus electrode that acts primarily as a cathode, sinking stimulus current, with the two neighbouring return electrodes on either side of the stimulus electrode acting primarily as anodes, sourcing return currents. Tripolar stimulus electrode configurations are described in more detail in International Patent Publication no. WO 2017/219096 by the present applicant, the entire contents of which are herein incorporated by reference.

In some implementations, the APM assumes that the electrode array 150 consists of two leads implanted approximately symmetrically to left and right (as viewed from behind the patient) of the patient's midline, as illustrated in FIG. 1 for one lead. In one implementation, each lead comprises twelve contacts (electrodes), numbered such that a contact index of zero is the topmost (rostral) contact of a lead and contact index 11 is the bottom-most (caudal) contact of a lead. The stimulus electrodes in each of the four candidate SECs are defined as follows: top left (contact index 1, left lead), top right (contact index 1, right lead), bottom left (contact index 10, left lead) and bottom right (contact index 10, right lead). In other implementations with a different number of contacts in each lead, the bottom left and bottom right stimulus electrodes are defined to be the second-most caudal contact on the respective leads.

In other implementations, the APM assumes other configurations for the electrode array 150. One such configuration is a paddle lead. In such an implementation, the stimulus electrodes in each of the four candidate SECs may be defined as the top left, top right, bottom left, and bottom right electrodes on the paddle lead.

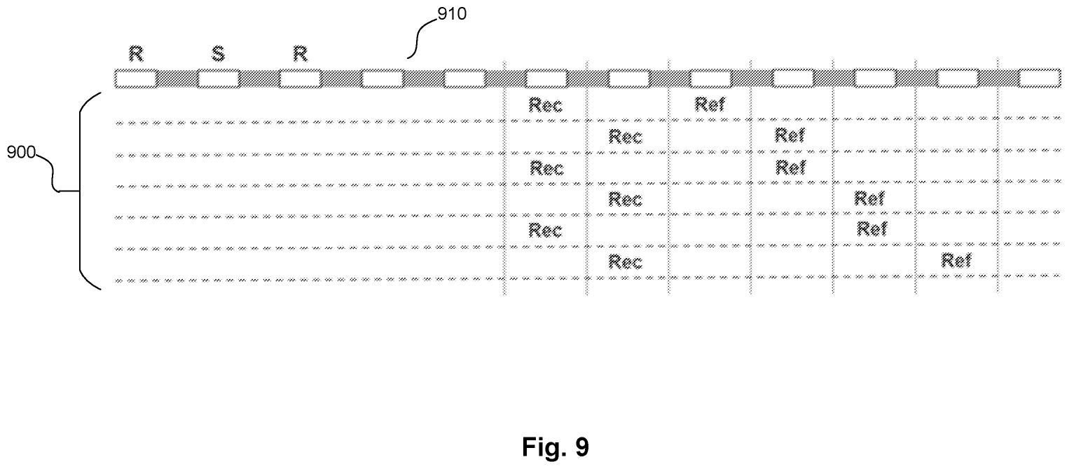

For each SEC, the APM defines multiple measurement electrode configurations (MECs). A measurement electrode configuration comprises two electrodes for differential ECAP recording, as illustrated in FIG. 3. The measurement electrode connected to the positive terminal of the measurement circuitry 318 is referred to as the recording electrode, while the measurement electrode connected to the negative terminal of the measurement circuitry 318 is referred to as the reference electrode. FIG. 9 illustrates the locations of the recording and reference electrodes in the six candidate MECs according to one implementation of the present technology. Each candidate MEC is represented in one row of the table 900 beneath a graphical representation 910 of a twelve-contact lead. The electrodes labelled Rec and Ref in each row are the recording and reference electrodes in the corresponding MEC. The electrodes labelled S and R are the stimulus and return electrodes of a tripolar SEC located, as described above, at one end of the lead.

In an alternative implementation of the present technology, the APM is provided with the patient's selected SECs by a means other than the stages 810 to 820. In such an implementation, the APM implements a workflow comprising only the measurement optimisation stage 830.

### Patient Controlled Stimulus Ramp Stage

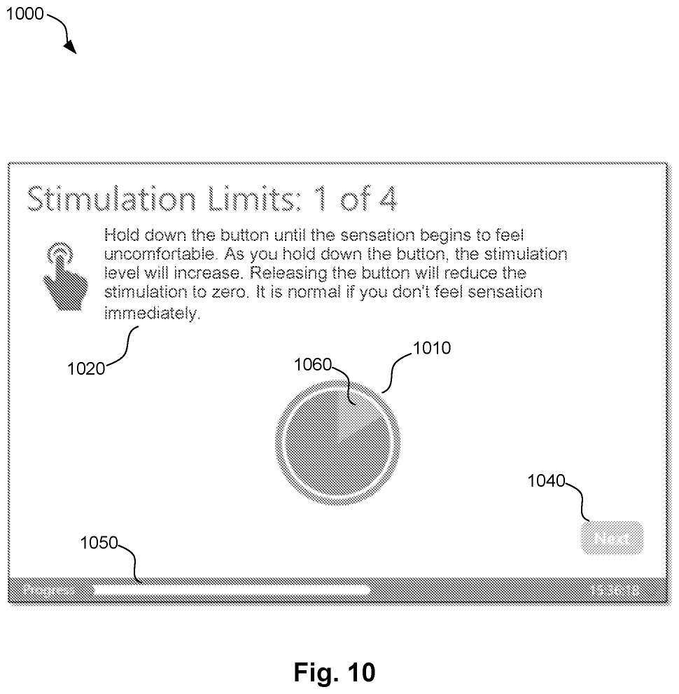

In one implementation of the PCSR stage 810, the APM renders on the UI display of the CI 740 a screen 1000 as illustrated in FIG. 10. The screen 1000 comprises a stimulation control 1010 (illustrated as a virtual button), a set of instructions 1020, a progress bar 1050, and a Next control 1040. The stimulation control 1010, once enabled, is configured to remain activated as long as the patient continues to interact with it, for example by “holding down” the virtual button. In other implementations of the PCSR stage 810, the stimulation control 1010 and/or the Next control 1040 are hardware controls, such as buttons, forming part of the UI of the CI 740 yet remaining separate from the display.

Once the stimulation control 1010 is enabled, the instructions 1020 are configured to instruct the patient to activate the stimulation control 1010. When the stimulation control 1010 is activated, the APM instructs the device 710 to deliver stimulation via the first of the candidate SECs at a gradually increasing or “ramping” intensity. The stimulation control 1010 may be animated to indicate the elapsed time since the activation of the control, for example by an animated “pie” display as illustrated in FIG. 10. In this example, a sector 1060 that represents the elapsed time is filled in a different manner to the remainder of the stimulation control 1010. While the stimulation control 1010 is activated, the sector 1060 grows wider in proportion to the elapsed time until it encompasses the entire stimulation control 1010. This animation indicates to the patient that something is happening when they activate the control 1010, even if they don't feel stimulation immediately (due to the stimulus intensity being below the perception threshold). The animation also conveys the rate of increase of stimulus intensity to the patient. The animation also indicates the stimulus intensity to a clinician or other skilled user. The animation also reinforces the instructions 1020. That is, even before the patient is able to feel stimulation, the patient can see the sector 1060 increasing when they activate the control 1010 and decreasing when they de-activate it.

In some implementations, the first activation of the stimulation control 1010 at a candidate SEC initiates a “pre-ramp” (described below). The pre-ramp is used to estimate the ECAP threshold Iₜₕᵣₑₛₕ for the candidate SEC, as described below. In such implementations, during the pre-ramp and/or the subsequent stimulus ramp at the same candidate SEC, the animation may indicate when the stimulus intensity has reached the ECAP threshold. The animation may indicate this by, for example, changing colour, or rendering an indicium on the display.

The APM continues to ramp the stimulus intensity as long as the patient continues to activate the stimulation control 1010. In one implementation of the stimulus ramp, the increase in intensity is linear with time with a predetermined ramp rate. The predetermined ramp rate may be set to 400 microamps/sec to minimise the risk of uncomfortable stimulation.

When the patient de-activates the stimulation control 1010, e.g. by releasing the virtual button, the APM records the stimulus intensity upon release as the Max value for the current SEC. The APM then ramps down the stimulus intensity. In one implementation, the down-ramp of intensity follows a linear profile, with the rate chosen such that the intensity reaches zero after a predetermined interval, for example three seconds.

The instructions 1020 encourage the patient to continue to activate the stimulation control 1010 for as long as is comfortable, ceasing the activation only when the intensity of stimulus begins to feel uncomfortable. This user interface design takes advantage of the human withdrawal reflex, whereby the patient is likely to instinctively release the button upon receiving uncomfortable stimulation. The design of the stage 810 therefore minimises the training burden placed on the patient in using the APM. If the patient does not cease to activate the stimulation control 1010 before the stimulus intensity reaches a hard ceiling (e.g. a pulse amplitude of 36 mA in one implementation), the APM ceases the stimulus ramp and begins a down-ramp. The stimulus intensity at the point of ceasing the stimulus ramp is recorded as the patient's discomfort threshold (Max) value for that SEC.

The progress bar 1050 indicates approximate quantitative progress through the workflow 800. In one implementation, the fraction of the progress bar 1050 that is filled in represents the current ratio of the elapsed time since the start of the workflow 800 to the average time taken to complete the workflow 800, as obtained from the assisted programming of previous patients according to the workflow 800.

Before and during each stimulus ramp, the APM collects and analyses data as described below. Following a successful stimulus ramp (as defined below), the Next control 1040 is enabled. On activation of the Next control 1040, a new stimulus ramp is carried out for the next candidate SEC. This cycle occurs once for each candidate SEC. Once all the candidate SECs have been used for a stimulus ramp, activation of the Next control 1040 moves the workflow 800 to the coverage selection stage 820.

Each stimulus ramp in the PCSR stage 810 is implemented by the APF on receipt of a ramp command from the APM. A ramp command specifies a ramp direction (up or down), a ramp rate (absolute change in intensity per unit of time), and an endpoint intensity. In one implementation, once the ramp command is received by the APF, the controller 116 initiates and continues the ramp until either the patient releases the stimulation control 1010, signalled to the APF by a Halt command from the APM, or the endpoint intensity is reached. Once the endpoint is reached, the APM sends a ramp-down command to the APF to ramp down the stimulus intensity. Because the purpose of the ramp is to determine the patient's Max value, the endpoint intensity is deliberately set high, i.e. above the highest expected Max value (in one implementation equal to 36 mA). This means that if for some reason communications between the APF and the APM are interrupted, the de-activation of the stimulation control 1010 will not be communicated to the APF, so according to this implementation there is a possibility the patient will receive uncomfortably intense stimulation until the APF ramps the stimulus intensity back down.

In another implementation, the controller 116 interrupts the ramp if the APF receives no communication from the APA within a first timeout period. The controller 116 may then ramp the intensity back down in the continued absence of communication from the APA within a second timeout period. In this implementation, the patient is less likely to receive uncomfortable stimulation if the communication between the APF and the APM is interrupted.

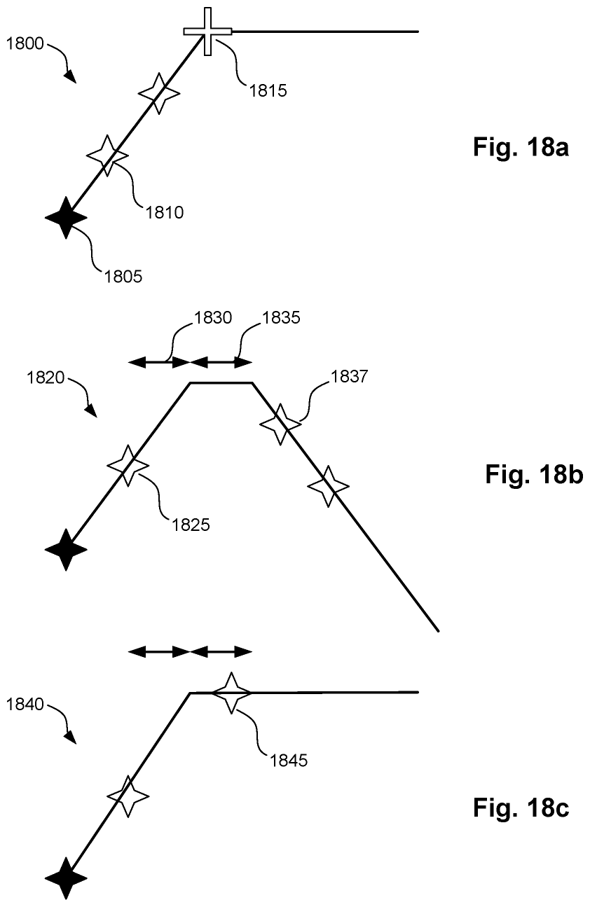

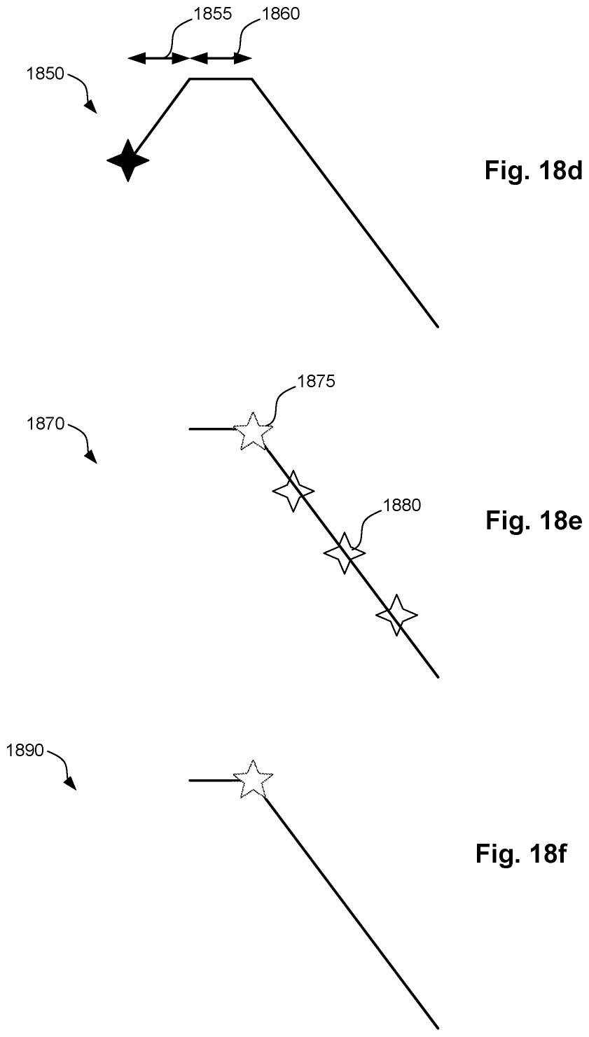

FIGS. 18a to 18f illustrate the operation of this implementation. In FIG. 18a, the ramp 1800 of stimulus intensity versus time is initiated on receipt by the APF of a Ramp command illustrated by the filled star 1805. The ramp 1800 continues as long as communications 1810 (illustrated by unfilled stars in FIG. 8) continue to be received by the APF. (The communications 1810 can be for any purpose, not just related to the PCSR.) The ramp 1800 halts when the APF receives a Halt command, illustrated by the cross 1815, from the APM. The ramp 1800 also halts if the endpoint intensity is reached (not illustrated).

The ramp 1820 in FIG. 18b occurs when communications are interrupted. After the ramp command and the communication 1825 are received, a first timeout period 1830 elapses with no further communications received by the APF. In one implementation, the first timeout period is one second. The ASPF therefore halts the ramp 1820. After the expiry of a second timeout period 1835 since the halt, the APF ramps down the intensity to zero, regardless of whether further communications, e.g. the communication 1837, are received during the down-ramp. In one implementation, the second timeout period is 0.5 seconds.

The ramp 1840 in FIG. 18c is halted prematurely for the same reason as in FIG. 18b. However, because the communication 1845 is received before the second timeout period has expired, the down-ramp does not take place.

In FIG. 18d, the ramp 1850 is halted prematurely due to the expiry of the first timeout period 1855. As in FIG. 18b, after the expiry of the second timeout period 1860 the APF ramps down the intensity to zero, regardless of the absence of communications from the APM.

FIG. 18e shows a down-ramp 1870 of intensity by the APF on receipt of a down-ramp command 1875 from the APM. The down-ramp 1870 continues to zero intensity, regardless of whether further communications, e.g. the communication 1880, are received during the down-ramp 1870.

The down-ramp 1890 in FIG. 18f, like the down-ramp 1870, continues to zero intensity regardless of the absence of communications from the APM during the down-ramp 1890.

### Data Analysis During PCSR Stage

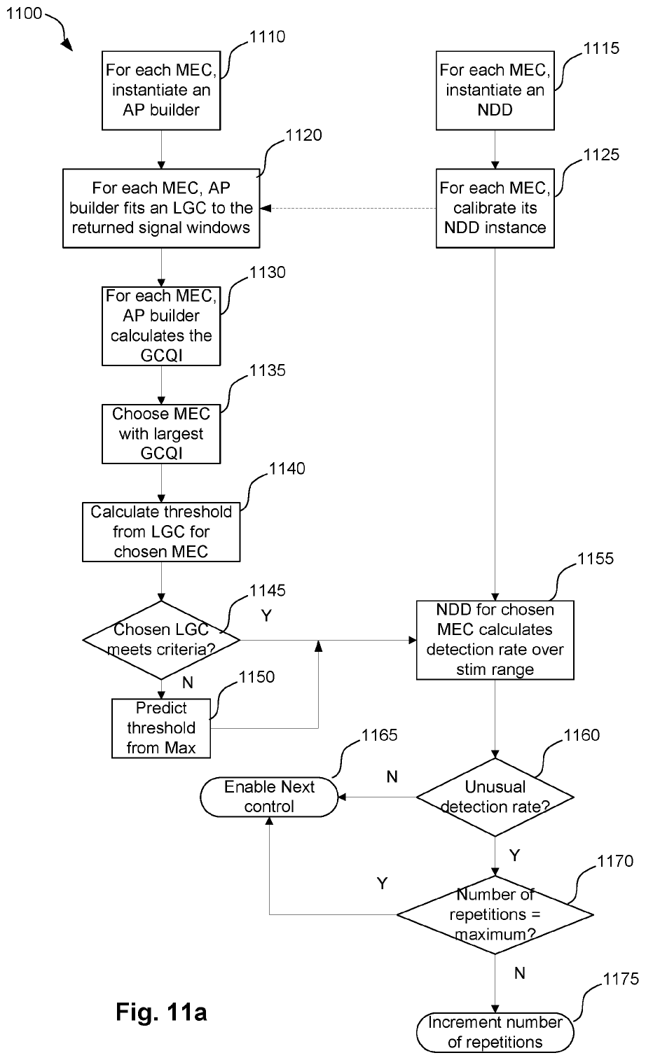

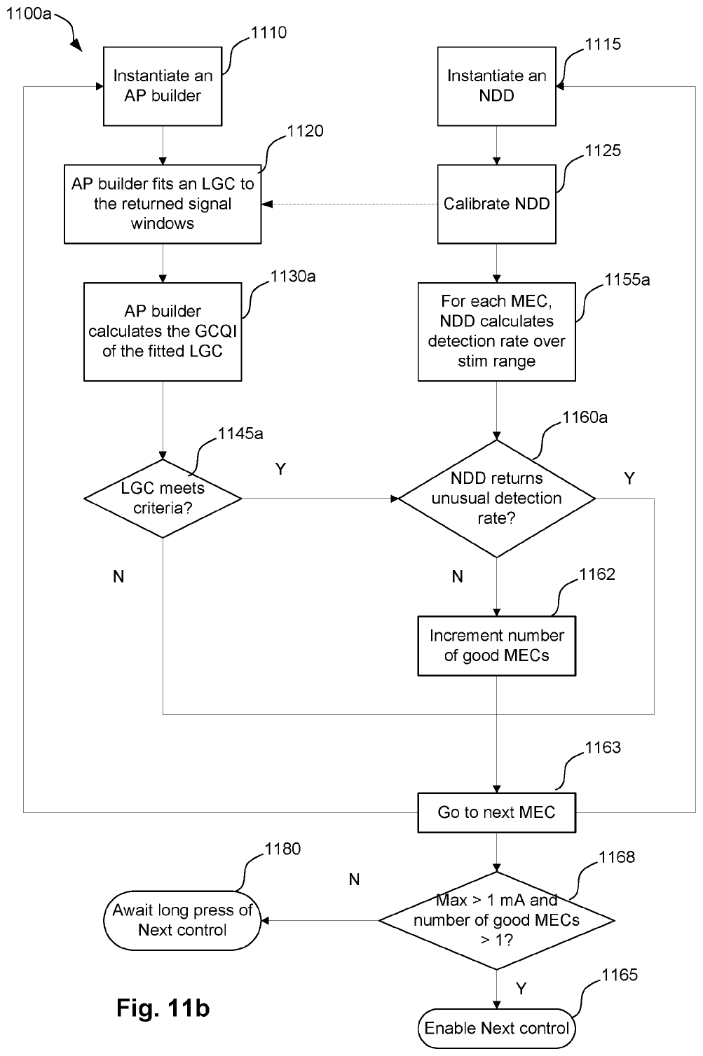

FIG. 11a is a flowchart illustrating a data collection and analysis method 1100 carried out by the APM and the device 710 during the PCSR stage 810 according to one implementation of the APM. The method 1100 is carried out for each stimulus ramp for each SEC. The method 1100 starts at steps 1110 and 1115. Steps 1110, 1115, and 1125 take place before the APM enables the stimulation control 1010 and therefore before any stimulus is applied. Step 1110 instantiates for each MEC an activation plot (AP) builder, while step 1115 instantiates for each MEC a noise departure detector (NDD). The AP builder and the NDD are described in more detail below.

At step 1125, the APM instructs the device 710 to capture multiple “zero current” signal windows for each MEC. In one implementation, the device 710 simply captures the signal windows using the measurement circuit 128 and bypasses the ECAP detector 320, storing the raw signal windows temporarily in memory 118 before transmitting the data to the APM. Once this data has been captured and returned to the APM, step 1125 processes these “zero current” signal windows to calibrate each NDD instance.

The method 1100 then proceeds to step 1120, which enables the stimulation control 1010 to allow the patient to commence the stimulus ramp for the current SEC as described above. During the stimulus ramp, the APM instructs the device 710 to capture and return signal windows at each MEC for each stimulus current amplitude s. The returned signal windows for each MEC are analysed by the corresponding AP builder, which extracts a detected ECAP amplitude d from each signal window. Once the stimulation control 1010 is de-activated, still at step 1120, each AP builder fits a model referred to as the Logistic Growth Curve (LGC) to the set of (s, d) value pairs for each MEC. Each AP builder then at step 1130 calculates a growth curve quality index (GCQI) for each fitted LGC. LGC model fitting and the calculation of the GCQI by the AP builder are described in more detail below.

Step 1135 then chooses the MEC which resulted in the largest GCQI. Step 1140 then calculates an ECAP threshold from the fitted LGC corresponding to the chosen MEC. Step 1140 is described in more detail below.

Step 1145 then tests whether the fitted LGC meets certain inclusion criteria:
The fitted LGC is based on more than a predetermined number (s, d) value pairs, e.g. 12 value pairs.
The GCQI is greater than a threshold, e.g. 10 dB.
The ECAP threshold calculated from the LGC is greater than 0 and less than the Max value recorded for the current SEC at the end of the stimulus ramp.

If any of the inclusion criteria are not met (“N”), the fitted LGC is disregarded, and the APM at step 1150 predicts the ECAP threshold Iₜₕᵣₑₛₕ from the Max value Iₘₐₓ recorded for the current SEC at the end of the stimulus ramp. In one implementation, step 1150 uses a linear prediction model:

$${I}_{thresh} = m \cdot {I}_{max} \qquad ( 3 )$$

where m is a correlation parameter that may be derived from historical patient data. In one implementation, m takes a value between 0.5 and 1.0. In another implementation, m takes a value between 0.6 and 0.9. In one implementation, m takes a value between 0.65 and 0.8. Step 1150 is an example of the prediction of a physiological threshold (the ECAP threshold) from a perceptual marker (the discomfort threshold, Max). The APM then proceeds to step 1155 using the predicted ECAP threshold.

If all the inclusion criteria tested at step 1145 are met (“Y”), the APM proceeds to step 1155 using the ECAP threshold value obtained at step 1140 from the fitted LGC model.

At step 1155, the APM uses the NDD to calculate a detection rate for the MEC chosen at step 1135 over a full range of stimulus intensity. In one implementation, the full range means a stimulus intensity between 1.1 times the ECAP threshold and the Max value. The detection rate is the proportion of stimulus intensity values over the full range for which the NDD returns greater than 50%. Step 1155 may use the signal windows returned during the stimulus ramp for the chosen MEC. Step 1160 then tests whether the detection rate is unusual. In one implementation, an unusual detection rate means a detection rate less than a predetermined fraction, for example 20%. The purpose of this test is to identify if the patient de-activated the stimulation control 1010 prematurely. This may occur if the patient is unfamiliar with the APM or if the patient de-activated the control accidentally.

If the detection rate is not unusual (“N”), the current SEC is marked as successful, and the method 1100 concludes at step 1165, at which the Next control 1040 is enabled. Otherwise (“Y”), step 1170 tests whether the maximum number of repetitions has been reached. If not (“N”), the APM at step 1175 increments the number of repetitions, and re-starts the method 1100 for the current candidate SEC. If so (“Y”), the current SEC is marked as unsuccessful, and the method 1100 concludes at step 1165, at which the Next control 1040 is enabled. As mentioned above, activation of the Next control 1040 either repeats the method 1100 for the next candidate SEC, or ends the PCSR stage 810 if all candidate SECs have been tested.

The result of the PCSR stage 810 is a Max value and an ECAP threshold for each candidate SEC marked as successful.

In other implementations of the PCSR stage 810:
The profile of the stimulus ramp during the activation of stimulation control 1010 may not be linear. One such implementation is a “threshold ramp”, with the threshold being the ECAP threshold, either predicted (as from step 1150) or fitted (as from step 1140). The threshold ramp is described in detail below.
The de-activation of the stimulation control 1010 may cause the stimulation intensity to be reduced exponentially rather than linearly. This handles the scenario where the perception of stimulation startles the patient, causing them to unintentionally release the stimulation control 1010. In such an implementation, the screen 1000 may include an additional user control to return the stimulus intensity all the way to zero in a controlled ramp and allow the patient to ‘lock in’ a Max value so that repetitions of the method 1100 may be handled. In another implementation, a threshold ramp is used for the down-ramp, with the threshold being the ECAP threshold, either predicted (as from step 1150) or fitted (as from step 1140). The threshold ramp is described in detail below.
The stimulus ramp rate may be increased or decreased based on the stimulation control de-activation point if the patient repeats the stimulus ramp for an SEC.
The search space of MECs may be extended from those illustrated in FIG. 9.
‘Early release’ and ‘missing ECAP’ failure scenarios may be distinguished, and different responses defined for each. For example, the patient may be asked whether they released the button by accident, and the method 1100 repeated as many times as necessary in that scenario.
One or more exclusion criteria, such as the detection of a late response, may be tested at step 1145 and if found to be true, used to exclude the fitted LGC.
There may be no maximum number of repetitions tested at step 1170. Instead, a “Y” at step 1160 leads straight to step 1175. If the calculation at step 1155 repeatedly results in an unusual detection rate for a candidate SEC, the Next control 1040 is therefore never enabled for that candidate SEC no matter how many times the method 1100 is repeated. In such a circumstance, holding down the Next control 1040 marks the candidate SEC as unsuccessful, and either repeats the method 1100 for the next candidate SEC, or ends the PCSR stage 810 if all candidate SECs have been tested.

FIG. 11b is a flowchart illustrating a data collection and analysis method 1100a carried out by the APM and the device 710 during the PCSR stage 810 according to one implementation of the APM. The method 1100a is carried out for each stimulus ramp for each SEC. The method 1100a is similar to the method 1100, in that steps that are the same in the two methods have like labels and as such are not described below. The main difference is that an MEC is not chosen midway through the method 1100a based on its GCQI. Instead, all quantities are computed for each MEC and the number of MECs whose computed quantities meet certain criteria are counted. If the count exceeds one, along with some other criteria, the Next control is enabled. Thus for example, at step 1155a, instead of computing the detection rate only for a chosen MEC, as at step 1155, the detection rate is computed for the current MEC in the list of candidate MECs. Also, at step 1130a, the AP builder for the current MEC calculates a growth curve quality index (GCQI) for the LGC fitted at step 1120. Step 1145a then tests whether the fitted LGC meets certain inclusion criteria. The purpose of the inclusion criteria of step 1145 is to confirm that the parameters fitted to the LGC can be trusted. The inclusion criteria are:
The GCQI is greater than a threshold, e.g. 6 dB.
The ECAP threshold calculated from the LGC is not too near the bounds of ECAP threshold within which the parameter fitting of the LGC took place.
The sensitivity calculated from the LGC is positive.
The standard deviation of the calculated sensitivity is less than a threshold, e.g. 0.5 times the calculated sensitivity.

If the fitted LGC does not meet the inclusion criteria (“N” at step 1145a), the method 1100a gets the next candidate MEC at step 1163 and returns to step 1110 and step 1115.

If the fitted LGC does meet the inclusion criteria (“Y” at step 1145a), the method 1100a checks, at step 1160a, whether the detection rate returned by the NDD for the current MEC at step 1155a is unusual, in the same sense as in step 1160. If the detection rate is not unusual (“N” at step 1160), or the GCQI is greater than 10 dB, the MEC may be deemed “good”. Step 1162 increments the number of “good” MECs at step 1163, and the method 1100a gets the next candidate MEC at step 1163 and returns to step 1110 and step 1115. If the detection rate is unusual (“Y” at step 1160), and the GCQI is less than or equal to 10 dB, the method 1100a proceeds directly to step 1163.

Once all the candidate MECs have been exhausted by step 1163, step 1168 tests whether the number of “good” MECs is greater than one, and the Max value is greater than a threshold, e.g. 1 mA. If so (“Y”), the method 1100a concludes by enabling the Next control at step 1165. If not (“N”), step 1180 waits for the user to “long press” (hold down for a predetermined interval) the Next control to end the method 1100a. If the method 1100a ends in this fashion, the current candidate SEC is marked as unsuccessful, meaning it takes no further part in the workflow 800.

### Coverage Survey Stage

In one implementation of the Coverage Survey stage 815, the APM renders on the UI display of the CI 740 a screen 1200 as illustrated in FIG. 12. The screen 1200 comprises a stimulation control 1210, a set of instructions 1220, a set of options 1230, a Next control 1240, and a progress bar 1250. In other implementations of the Coverage Survey stage 815, the stimulation control 1210 and/or the Next control 1240 are hardware controls, such as buttons, forming part of the UI of the CI 740 yet remaining separate from the display.

The screen 1200 is rendered at least once for each successful candidate SEC from the PCSR stage 810 to implement a Coverage Survey for that SEC. The stimulation control 1210 is in the form of a tile that, upon activation by the user, toggles stimulation on and off via the current candidate SEC. In one implementation of the coverage survey stage 815, stimulation turns on and off at the current candidate SEC by threshold ramps to and from a comfortable stimulus intensity for the current candidate SEC, as estimated at the PCSR stage 810. The threshold for the threshold ramp is the ECAP threshold for the current candidate SEC, as estimated at the PCSR stage 810. Threshold ramps are described below.

An initial comfortable stimulus intensity for each candidate SEC may be predicted at the start of the coverage survey for that candidate SEC from the Max value Iₘₐₓ and the ECAP threshold Iₜₕᵣₑₛₕ that were estimated for the candidate SEC at the PCSR stage 810. In one implementation, the comfortable stimulus intensity I_{comf} may be calculated as a fixed proportion of the interval between Iₜₕᵣₑₛₕ and Iₘₐₓ for the candidate SEC:

$${I}_{comf} = {I}_{thresh} + k ( {I}_{max} - {I}_{thresh} ) \qquad ( 4 )$$

where k is a predetermined constant between 0 and 1. This prediction is an example of the prediction of a perceptual marker (the comfortable stimulus intensity) from a physiological threshold (the ECAP threshold).

In an alternative implementation, the ECAP threshold Iₜₕᵣₑₛₕ is estimated for the candidate SEC at the PCSR stage 810 using a “pre-ramp” (described below). The comfortable stimulus intensity I_{comf} may be calculated directly from Iₜₕᵣₑₛₕ by inverting the linear model of Equation (3) and substituting the result into Equation (4) in place of the Max value Iₘₐₓ. In such an implementation, the Max value Iₘₐₓ does not need to be determined during the PCSR stage.

The instructions 1220 instruct the user to activate the stimulation control 1210 and to select one or more of the options 1230 to provide feedback about their sensations. Each option 1230 corresponds to a line of text next to a circular control. The APM then waits for the patient to select one or more of the options 1230 and activate the Next control 1240. The Next control 1240 is disabled until stimulation has been tested and least one option is selected. An option may be selected or deselected by activating the control next to its text.

In some implementations, for each candidate SEC, the options 1230 are not displayed until after the user has activated the stimulation control corresponding to that SEC.

The progress bar 1250 at the bottom of the screen 1200, like the progress bar 1050, indicates approximate quantitative progress through the entire workflow 800.

Once the Next control 1240 is activated, the APM responds to the options selected for the current candidate SEC with a “mitigation” selected according to Table 1. A “1” in a column of Table 1 represents the selection of the option corresponding to that column, a “0” represents non-selection, and an “X” means either the option was selected or not (the selection of the option does not affect the chosen mitigation).

TABLE 1
Mitigations in first iteration of
Coverage Survey for a candidate SEC

| | Too | | | |
|---|---|---|---|---|
| Uncomfortable | strong | Too weak | Feels fine | Mitigation |
| 0 | 0 | 0 | 0 | N/A |
| 0 | 0 | 0 | 1 | None |
| 0 | 0 | 1 | X | Increase comfortable |
| | | | | stimulus intensity |
| 0 | 1 | X | X | Decrease comfortable |
| | | | | stimulus intensity |
| 1 | X | X | X | Move candidate SEC |
| | | | | to a new location |

The mitigations to increase and decrease the comfortable stimulus intensity do so by a small amount, equal to 0.05×(Iₘₐₓ−Iₜₕᵣₑₛₕ) in one implementation. However, the decrease and increase mitigations are not permitted to move the comfortable stimulus intensity outside the therapeutic range defined as [Iₘₐₓ, Iₜₕᵣₑₛₕ]. If the comfortable stimulus intensity is adjusted according to these mitigations, the Coverage Survey stage 815 may then be repeated for the adjusted comfortable stimulus intensity.

The mitigation to move the current candidate SEC does so by one electrode towards the middle of the lead. If the current candidate SEC is moved according to this mitigation, a PCSR (described above) may be repeated for the relocated candidate SEC. The Coverage Survey stage 815 is then repeated for the relocated candidate SEC.

In some implementations, for each candidate SEC, the “too weak” and/or the “feels fine” options are not enabled until the control 1210 has been activated for long enough for the stimulation intensity to ramp up to the comfortable stimulus intensity. This prevents the patient from responding to the Coverage Survey with incomplete information.

If the Coverage Survey is repeated for a candidate SEC, the APM responds to selections for that candidate SEC with a mitigation selected according to Table 2. As in Table 1, a “1” in a column of Table 2 represents the selection of the option corresponding to that column, a “0” represents non-selection, and an “X” means either the option was selected or not (the selection of the option does not affect the chosen mitigation.

TABLE 2
Mitigations in second iteration of
Coverage Survey for a candidate SEC

| | Too | | | |
|---|---|---|---|---|
| Uncomfortable | strong | Too weak | Feels fine | Mitigation |
| 0 | 0 | 0 | 0 | N/A |
| 0 | 0 | 0 | 1 | None |
| 0 | 0 | 1 | X | Increase comfortable |
| | | | | stimulus intensity |
| 0 | 1 | X | X | Decrease comfortable |
| | | | | stimulus intensity |
| 1 | X | X | X | Decrease comfortable |
| | | | | stimulus intensity |

In some implementations of the workflow 800, a PCSR may only be repeated once (i.e. iterated twice) for any candidate SEC, to reduce the burden on the patient of repeatedly having to undergo PCSRs with a relocated SEC.

If the patient still feels discomfort in certain areas for a candidate SEC at the second iteration of the Coverage Survey for that candidate SEC, the comfortable stimulus intensity for that candidate SEC is decreased (as per the final row of Table 2). In an alternative implementation, that candidate SEC is marked as unsuccessful. The Coverage Survey is not repeated for that candidate SEC.

The Coverage Survey stage 815 ends with a set of successful candidate SECs and their respective notional comfortable stimulus intensities. If the patient still feels discomfort in certain areas for a candidate SEC after the second iteration of the Coverage Survey stage 815, the patient will have the opportunity to discard that candidate SEC during the Coverage Selection stage 820.

### Noise Departure Detector (NDD)

The NDD is a statistical detector of the presence of an ECAP in a signal window. The operation of the NDD on a signal window is preferably preceded by an “artefact scrubber” which removes artefact from the signal window. One such artefact scrubber is disclosed in International Patent Publication no. WO 2020/124135, the entire contents of which are herein incorporated by reference. The NDD works by detecting a statistically unusual difference from the expected noise present in a signal window, which difference is attributed to the presence of an ECAP in the signal window.

The calibration of an NDD instance corresponding to an MEC, which occurs for example during step 1125 of the method 1100, may be carried out on one or more signal windows captured via that MEC which are known not to contain evoked neural responses. In one implementation, such signal windows are “zero current” signal windows which are captured from intervals during which no stimulus is being applied, and which have preferably been scrubbed for artefact, and may therefore be treated as comprising only noise. The calibration comprises forming estimates of parameters of a predetermined “noise model” (statistical distribution) from the samples in the one or more “zero current” signal windows. In one implementation, the noise model is Gaussian and the parameters are the mean \tilde{μ} and standard deviation ô of the samples.

Once calibrated, an NDD instance may be applied to a signal window (as in step 1155 of the method 1100) by counting the number \tilde{k} of outliers in the signal window, i.e. the number of samples in the signal window that depart significantly from the noise model. For a Gaussian noise model, the NDD counts the number \tilde{k} of samples that differ from the mean estimate \tilde{μ} by more than n times the standard deviation estimate \hat{σ}, where n is a small integer. The number \tilde{k} of outliers is compared to the number k of samples that would be expected to occur if the signal window consisted solely of noise with mean \tilde{μ} and standard deviation \hat{σ}. The difference between \tilde{k} and k is divided by the number of samples N in the signal window to obtain a metric r that quantifies the ratio of outliers present in a signal window relative to the expected ratio of outliers in a signal window that obeys the noise model.

It may be shown that for Gaussian noise model, the NDD may estimate the metric r as

$$r = \frac{\tilde{k}}{N} - 2 Φ ( \frac{n}{2} ) \qquad ( 5 )$$

where Φ is the standard normal cumulative distribution function.

A negative or zero value of the metric r indicates a signal window consistent with the noise model, whereas a positive value of r indicates a departure from the noise model. Such a departure is deemed to be due to the presence of an ECAP in the signal window.

In one implementation of the NDD, n is set to 3. Smaller values of n make the NDD more sensitive, indicating a departure from noise more readily and increasing the rate of Type I errors (false positives). Conversely, high values for n necessitate large outliers before r will indicate a noise departure, increasing the rate of Type II errors (false negatives).

In one implementation of the NDD, a sigmoid function may be applied to the raw metric r to map the metric r to a quality indicator Q_{NDD} in the interval [0, 1]:

$${Q}_{NDD} = \frac{1}{1 + exp ( - γ r )} \qquad ( 6 )$$

where γ is a parameter that balances the Type I and Type II errors. The quality indicator Q_{NDD} has a natural interpretation: Q_{NDD}<0.5 corresponds to r≤0 and indicates that the signal window is most likely noise. Conversely, Q_{NDD}>0.5 indicates a departure from the noise model that is deemed to represent an ECAP. In one implementation, γ is set to 50.

In one implementation, the NDD may be applied to multiple signal windows after they have been averaged together to improve the signal-to-noise ratio. In one such implementation, the number of averaged signal windows is eight. In such implementations, the parameters of the noise model may be adjusted depending on the number of signal windows that are averaged. In the Gaussian noise model, the standard deviation \hat{σ} should be divided by the square root of the number of averaged signal windows.

The NDD may be used to estimate the ECAP threshold. In one such implementation, the ECAP threshold is the stimulus intensity at which the NDD returns a quality indicator of 50% (0.5), i.e. at which the NDD detects an ECAP in 50% of signal windows processed. In one implementation, the ECAP threshold may be located during a ramp of stimulus intensity while monitoring the quality indicator Q_{NDD}. As soon as the quality indicator Q_{NDD} consistently exceeds 50%, the ECAP threshold has been reached.

This use of the NDD to estimate the ECAP threshold may be employed at an alternative implementation of step 1150.

This use of the NDD may also be employed in an alternative implementation of the PCSR stage 810. In such an alternative implementation, the ramp rate of stimulus intensity while the stimulation control 1010 is activated is not predetermined, but is calculated from the results of a “pre-ramp”. During the pre-ramp, which commences when the stimulation control 1010 is activated, the NDD is used to estimate the ECAP threshold as described above. The pre-ramp ends by ramping the stimulus intensity down to zero. The value of Max is then predicted from the ECAP threshold estimated during the pre-ramp. This prediction step, which is an example of the prediction of a perceptual marker from a physiological threshold, may be implemented by inverting the linear model of Equation (3). A ramp rate is then calculated such that the patient-controlled stimulus ramp would reach the predicted value of Max after a predetermined time. The calculated ramp rate is then used for the patient-controlled stimulus ramp which takes places as described above on the next activation of the stimulation control 1010.

### AP Builder

As mentioned above, the AP builder, as used for example at step 1120 of the method 1100, fits a model referred to as the Logistic Growth Curve (LGC) to a set of (s, d) value pairs, where dis a measured ECAP amplitude from a signal window and s is the corresponding stimulus current amplitude. The AP builder may also, for example at step 1130 of the method 1100, calculate a growth curve quality index (GCQI) for a fitted LGC.

An important part of the AP builder is an ECAP detector that returns the ECAP amplitude d from a signal window. In one implementation, the ECAP detector described in the above-mentioned International Patent Publication no. WO 2020/124135 may be used by the AP builder to measure the amplitude d of the ECAP in a signal window. Alternatively, the ECAP detector described in the above-mentioned International Patent Publication no. WO 2015/074121 may be used by the AP builder to measure the amplitude d of the ECAP in a signal window. In both cases, the ECAP detector has two parameters: its correlation delay, and its length (or equivalently its frequency). Other implementations of ECAP detectors may have other adjustable parameters. The optimal values of these parameters are dependent on the SEC and the MEC that gave rise to the signal window and should therefore be tuned for each instance of the AP builder, for example the six instances instantiated at step 1110 of the method 1100. In one implementation, the AP builder may tune the ECAP detector parameters on an average signal window obtained by averaging the ten signal windows corresponding to the largest values of stimulus current amplitude s. In one implementation, the above-described NDD may first be applied to each signal window before incorporating it into the average signal window. If the NDD indicates that the signal window did not contain a neural response, the signal window is discarded.

The ECAP detector is applied to the average signal window for every feasible value of correlation delay and length to form a correlation matrix. In one example of tuning the parameters of an ECAP detector, the values of correlation delay and length that maximise the measured ECAP amplitude within the correlation matrix are chosen as optimal for that instance of the AP builder. During a stimulus ramp, as the stimulus current increases, the AP builder may dynamically update the optimal values of correlation delay and length using the most recent average signal window. The AP builder may retrospectively recalculate ECAP amplitudes for all signal windows captured since the start of the current stimulus ramp using the currently optimal values.

Once the ECAP detector has been tuned and the set of (s, d) value pairs has been obtained, the AP builder proceeds to fit an LGC model (also referred to as a sigmoid function) to the set of (s, d) value pairs. In one implementation, the LGC model is a four-parameter function:

$$d ( s ) = A + \frac{K - A}{1 + exp ( - B ( s - M ) )} \qquad ( 7 )$$

where the four parameters are:
A, the minimum value (the detected ECAP amplitude in the absence of stimulation)
K, the maximum value (the detected ECAP amplitude at which saturation occurs, i.e. increases in stimulus intensity do no increase the detected ECAP amplitude)
M, the current amplitude at the midpoint between A and K.
B, the steepness of the LGC, which is proportional to the gradient at the midpoint between A and K.

In other implementations, fewer parameters may be used for the LGC model, for example an LGC model in which the minimum value A is identically zero. In yet other implementations, other parametrised functions may be fit by the AP builder to the set of (s, d) value pairs.

To fit the LGC, the parameters A, K, M, and B may be initialised to sensible starting points A₀, K₀, M₀, and B₀. In one implementation, these values may be set to:
A₀: the mean of the ECAP amplitudes obtained from the lowest few stimulus current amplitudes.
K₀: the mean of the ECAP amplitudes obtained from the highest few stimulus current amplitudes.
M₀: the stimulus current amplitude at the midpoint between A and K
B₀: may be calculated from the gradient m at the midpoint, obtained from local linear regression of value pairs acquired near the midpoint, as B₀=m*4/(K₀−A₀).

An optimisation algorithm such as Trust Region Reflective (TRF) may then be used to optimise the four parameters A, K, M, and B from their starting points A₀, K₀, M₀, and B₀.

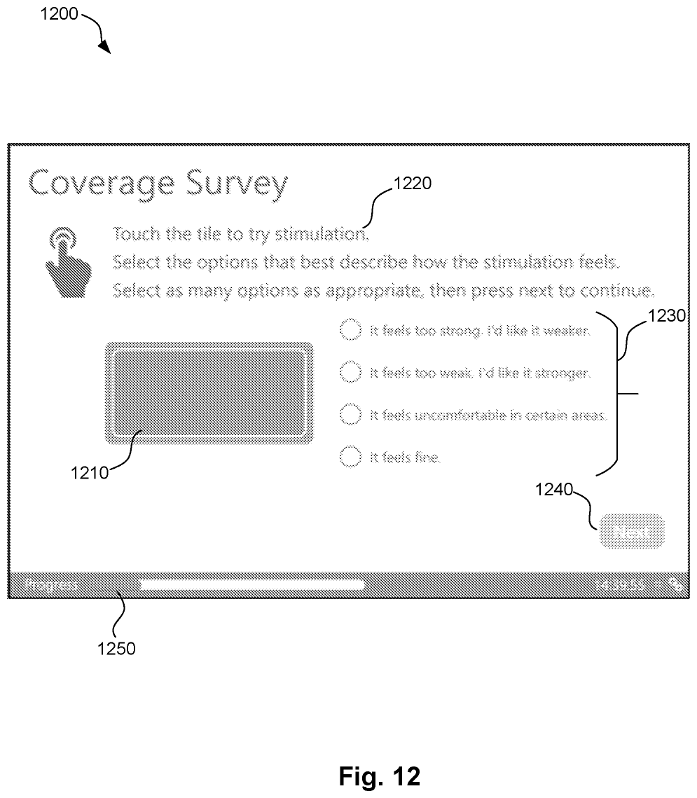

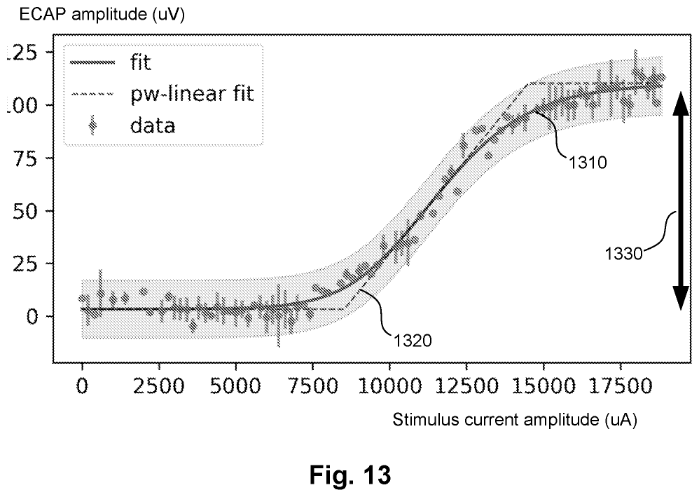

FIG. 13 shows a fitted LGC model 1310 to a set of (s, d) value pairs, alongside a piecewise linear model 1320 fit to the same data. The superior fit of the LGC model to the data at both low and high stimulus current amplitudes is evident.

The AP builder may also, for example at step 1130 of the method 1100, calculate a growth curve quality index (GCQI) for the fitted LGC model. The GCQI indicates a signal-to-noise ratio (SNR) of the fitted LGC. In one implementation, the AP builder may calculate the GCQI by dividing the peak-to-peak amplitude of the fitted LGC (e.g. as indicated in FIG. 13 by the arrow 1330) by the standard deviation of the residuals of the fitted LGC.

The fitted LGC may be used to estimate the ECAP threshold Iₜₕᵣₑₛₕ, as in step 1140 of the method 1100 or step 1740 of the method 1700 (described below). In one implementation, a line is constructed through the midpoint M of the fitted LGC with slope B. The ECAP threshold Iₜₕᵣₑₛₕ may be estimated as the stimulus current amplitude s at which the constructed line intersects the minimum value A. It may be shown that the resulting ECAP threshold Iₜₕᵣₑₛₕ is given by:

$${I}_{thresh} = M - \frac{2}{B} \qquad ( 8 )$$

The fitted LGC may be used to estimate the patient sensitivity, as in step 1740 of the method 1700. In one implementation, the patient sensitivity S is the slope of the fitted LGC at its midpoint M, which may be computed from the steepness B as follows:

$$S = \frac{B}{4} ( K - A ) \qquad ( 9 )$$

The fitted LGC may be used to estimate the discomfort threshold, Max, in another example of the prediction of a perceptual marker (the discomfort threshold, Max) from a physiological threshold. In this example, the physiological threshold is the stimulus current amplitude at which the LGC model saturates, i.e. the saturation threshold. In one implementation, saturation may be said to have occurred when d(s) reaches A+U (K−A), where U is just less than one. The corresponding value sₛₐₜ of the saturation threshold may be computed as:

$${s}_{sat} = M - \frac{1}{B} log ( \frac{1}{U} - 1 ) \qquad ( 10 )$$

The discomfort threshold, Max, may then be estimated from the saturation threshold by a linear predictive model.

### Threshold Ramp

A threshold ramp is a ramp of stimulus intensity, either up or down, that traverses stimulus intensity values below a predetermined threshold value at a faster rate than the ramp traverses stimulus intensity values above the predetermined threshold value.

When ramping stimulus intensity up, it is preferred by patients that the ramp feel gradual rather than abrupt. However, it is also generally desirable to produce a user interface that feels responsive to the patient. For example, during the PCSR stage 810, the patient may de-activate the stimulation control 1010, causing the stimulation to turn off. If they do so in response to an uncomfortable stimulus, the responsiveness of the user interface is important. A patient will be more willing to experiment with their comfort limits if stimulation ramps down quickly without producing discomfort.

Stimulus intensities below the ECAP threshold are generally not perceivable by patients. Therefore, ramping through sub-ECAP-threshold intensities does not improve the patient's sensation of gradualness and may in fact detract, by taking up unnecessary time, from the patient's sensation of responsiveness. A threshold ramp may therefore skip over most sub-ECAP-threshold stimulus intensities on either the way up or the way down.

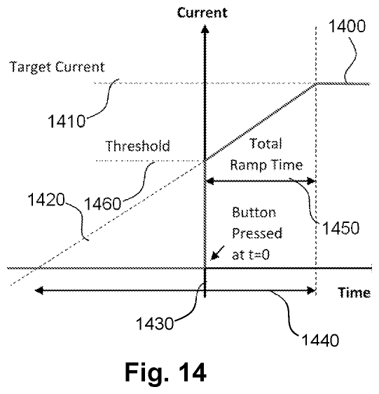

FIG. 14 illustrates a threshold ramp according to one implementation of the present technology. The profile 1400 represents the time course of stimulus current amplitude according to a threshold ramp up to a target current amplitude 1410. The dotted profile 1420 represents the time course of stimulus current amplitude according to a conventional linear ramp from zero to the target current amplitude 1410. The instant 1430 represents the time (t=0) at which the ramp was initiated, e.g. by activation of the stimulation control 1010. The interval 1440 represents the predetermined time that would have been taken by the conventional linear ramp, for example three seconds, to reach the target current amplitude 1410. The ramp rate of the conventional linear ramp profile 1420 is calculated such that the stimulus intensity reaches the target current amplitude 1410 at the end of the interval 1440. The threshold ramp, by contrast, steps comparatively rapidly (e.g. vertically) to a threshold current amplitude 1460. Then during the interval 1450, the threshold ramp linearly increases the stimulus current amplitude at the same rate as the conventional linear ramp. The length of the interval 1450, i.e. the total ramp time, is therefore significantly less than the predetermined time of the interval 1440. The threshold ramp therefore appears more responsive to the patient. Moreover, if the threshold current amplitude 1460 is set slightly below the ECAP threshold, the threshold ramp does not appear any more abrupt than the conventional linear ramp, since the patient is unable to perceive stimulus current amplitudes below the threshold current amplitude 1460.

In one implementation, the threshold current amplitude 1460 may be obtained by scaling the ECAP threshold by 0.9. This scaling factor provides a balance between having faster overall ramp times and keeping the likelihood of a step to a perceptible current amplitude low.

A threshold down-ramp according to one implementation is a time-reversed version of the profile 1400 of the threshold ramp illustrated in FIG. 14. In other words, a threshold down-ramp from a starting current amplitude decreases current amplitude linearly at a rate equivalent to a conventional linear down-ramp over the predetermined interval 1440. When the stimulus current amplitude reaches the threshold current amplitude 1460, the stimulus current amplitude steps comparatively rapidly (e.g. vertically) to zero.

In other implementations of the threshold ramp, the profile of stimulus current amplitude is not piecewise linear as in FIG. 14. Instead, alternative profiles of stimulus intensity may be used. The alternative profiles are also parametrised by a threshold value. In one such implementation, the profile follows a sigmoid function, such as described above, that smoothly and exponentially rises from zero to a midpoint that is computed from the threshold, and decelerates as the stimulus current amplitude approaches the target current amplitude. Another such implementation is an exponential profile below the threshold, followed by a linear profile above the threshold. The ramp rate of the linear profile is chosen to be less than the average ramp rate of the exponential profile.

In some implementations, as described above in relation to the Patient-Controlled Stimulus Ramp stage 810, a threshold ramp may be interrupted if the APF receives no communication from the APA within a first timeout period. The controller 116 may then ramp the intensity back down in the continued absence of communication from the APA within a second timeout period. Example profiles of such implementations of a threshold ramp are illustrated in FIGS. 18a to 18c. In such implementations, the patient is less likely to receive uncomfortable stimulation if the communication between the APF and the APM is interrupted.

### Coverage Selection Stage

As mentioned above, the coverage selection stage 820 is configured to receive input from the patient to select one or more of the successful candidate SECs from the coverage survey stage 815, based on the Max and ECAP threshold values for that candidate SEC. The patient can test different combinations of candidate SECs before selecting which ones to keep.

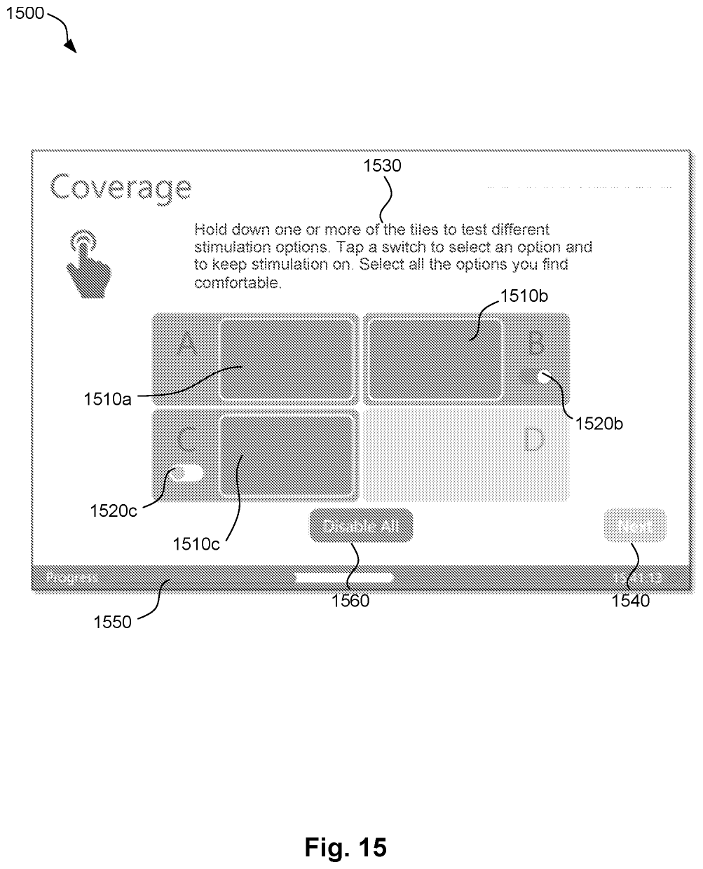

In one implementation of the coverage selection stage 820, the APM renders on the UI display of the CI 740 a screen 1500 as illustrated in FIG. 15. The screen 1500 comprises controls comprising: up to four toggle tiles, e.g. 1510a, 1510b, and 1510c, up to four toggle switches, e.g. 1520b and 1520c, a Next control 1540, a progress bar 1550, and a Disable All control 1560.

Toggle switches 1520b and 1520c are associated with respective toggle tiles 1510b and 1510c. However, toggle tile 1510a has no associated toggle switch in FIG. 15. This is because the switch corresponding to a tile is not rendered until the tile has been activated once. In the state of the coverage selection stage 830 illustrated in FIG. 15, tile 1510a has not yet been activated, so tile 1510a has no associated switch. However, tiles 1510b and 1510c have been activated, so tiles 1510b and 1510c have associated switches 1520b and 1520c.

In other implementations of the coverage selection stage 820, one or more of the controls are hardware controls, such as buttons or switches, forming part of the UI of the CI 740 yet remaining separate from the display. The UI also comprises instructions 1530.

Each toggle control pair, e.g. the tile 1510b and the switch 1520b, corresponds to one of the successful candidate SECs after the coverage survey stage 815. (As an example, only three control pairs are shown in FIG. 15, as the fourth candidate SEC was marked as unsuccessful during the PCSR stage 810.) The four (tile, switch) control pairs may be activated and de-activated independently. The state of stimulation on an SEC (on or off) corresponds to the state of the corresponding toggle switch (activated or de-activated). The stimulus pulses from all the “on” SECs at a given time are interleaved in a predetermined time order, staggered in time by the inter-stimulus interval.

Each toggle tile is configured to remain activated as long as the patient continues to interact with it, for example by “holding down” the toggle tile, and becomes de-activated when the patient ceases to interact with it, for example by “releasing” the toggle tile. The toggle tile takes on a different appearance when it is activated, for example by being filled in a different colour. By contrast, each toggle switch cannot be “held down”, but inverts its state from de-activated to activated or from activated to de-activated each time the patient interacts with the toggle switch. The toggle switch takes on a different appearance when it is activated, for example by filling in the disk representing the toggle switch.

In one implementation, the toggle tiles have an inverting behaviour, whereby for as long as the toggle tile is being activated, e.g. held down, the state of stimulation, which is always indicated by the state of the toggle switch, is inverted. For example, if the toggle switch is activated, activating the corresponding tile de-activates the toggle switch and stops stimulation, and de-activating the tile activates the toggle switch and restarts stimulation. Conversely, if the toggle switch is de-activated, holding down the corresponding tile activates the toggle switch and starts stimulation, and releasing the tile de-activates the toggle switch and stops stimulation. The stimulation is always on if the switch is activated, and always off if the switch is de-activated. The appearance of a toggle switch therefore offers a visual cue to indicate the state of stimulation on the corresponding SEC.

Table 3 summarises the effect of activating and de-activating the toggle tile and the toggle switch on the stimulation from the corresponding candidate SEC according to this implementation of the coverage selection stage 820. Blank cells represent actions that cannot occur.

TABLE 3
State transition table for one implementation
of coverage selection stage

| | Stimulation/ | Activate | De-activate | Activate | De-Activate |
|---|---|---|---|---|---|
| State No. | Switch state | tile | tile | switch | switch |
| 1 | Off/De- | 2 | 2 | 2 | |
| | activated | | | | |
| 2 | On/ | 1 | 1 | | 1 |
| | Activated | | | | |

In another implementation, if the toggle switch is activated, activating the corresponding tile de-activates the toggle switch and stops stimulation, and de-activating the tile does not further change the state of stimulation. Conversely, if the toggle switch is de-activated, holding down the corresponding tile activates the toggle switch and starts stimulation, and releasing the tile de-activates the toggle switch and stops stimulation. Table 4 Table 3 summarises the effect of activating and de-activating the toggle tile and the toggle switch on the stimulation from the corresponding candidate SEC according to this implementation of the coverage selection stage 820.

TABLE 4
State transition table for alternative implementation
of coverage selection stage

| | Stimulation/ | Activate | De-activate | Activate | De-Activate |
|---|---|---|---|---|---|
| State No. | Switch state | tile | tile | switch | switch |
| 1 | Off/De- | 2 | 1 | 2 | |
| | activated | | | | |
| 2 | On/ | 1 | 1 | | 1 |
| | Activated | | | | |

Under the implementation summarised in Table 4, the behaviour of stopping stimulation when a stimulus control is de-activated, as during the PCSR and coverage survey stages, is maintained.

The progress bar 1550, like the progress bars 1050 and 1250, indicates approximate quantitative progress through the entire workflow 800.

The Disable All control 1560 disables all stimulation and de-activates all toggle switches 1520b etc.

The instructions 1530 inform the patient that when they activate (“hold down”) a toggle tile, they will feel stimulation in one of four locations.

In an alternative implementation of the coverage selection stage 820, there are no toggle tiles, only toggle switches.

In one implementation of the coverage selection stage 820, stimulation turns on and off at a candidate SEC by threshold ramps to and from the comfortable stimulus intensity for the candidate SEC that resulted from the Coverage Survey stage 815. The threshold for the threshold ramp is the ECAP threshold for the candidate SEC that was estimated at the PCSR stage 810. Threshold ramps are described above.

The Next control 1540 is enabled as long as at least one toggle switch is activated. In some implementations, an additional criterion for enabling the Next control 1540 is that stimulation according to the final selected coverage needs to have been active for a minimum duration, for example five seconds. Once the patient activates the Next control 1540, the APM records the currently activated candidate SECs as the selected SECs, and stimulation is stopped on all SECs.

In an alternative implementation of the coverage selection stage 820, there are no toggle switches, only toggle tiles.

In such an implementation, the Next control 1540 is enabled as long as at least one toggle tile is activated. Once the patient activates the Next control 1540, the APM records the currently activated candidate SECs as the selected SECs, and stimulation is stopped on all SECs.

### Measurement Optimisation Stage

As mentioned above, the Measurement Optimisation (MO) stage 830 is configured to deliver stimulus of a gradually increasing intensity from a primary SEC of the selected SECs, and record sensed signal data at each of multiple measurement electrode configurations for the primary SEC. The MO stage 830 is then configured to choose the optimal MEC for the primary SEC, calculate physiological characteristics of the patient based on the neural responses extracted from signal windows recorded via the optimal MEC, and choose optimal therapy parameters for the primary SEC/optimal MEC combination.

The primary SEC in the determined program is the selected SEC from which neural responses are measured to drive the feedback loop to adjust the stimulus current amplitude of the primary SEC in accordance with the system 300 as described above. Neural responses evoked by the non-primary selected SECs are not recorded or analysed. Instead, the stimulus current amplitudes of the non-primary SECs are adjusted by the controller 116 so they remain in fixed ratios with the stimulus current amplitude of the primary SEC. The ratios to which the non-primary selected SECs are fixed may be saved in the determined program as the ratios of their respective comfortable stimulus intensities to the comfortable stimulus intensity of the primary SEC.

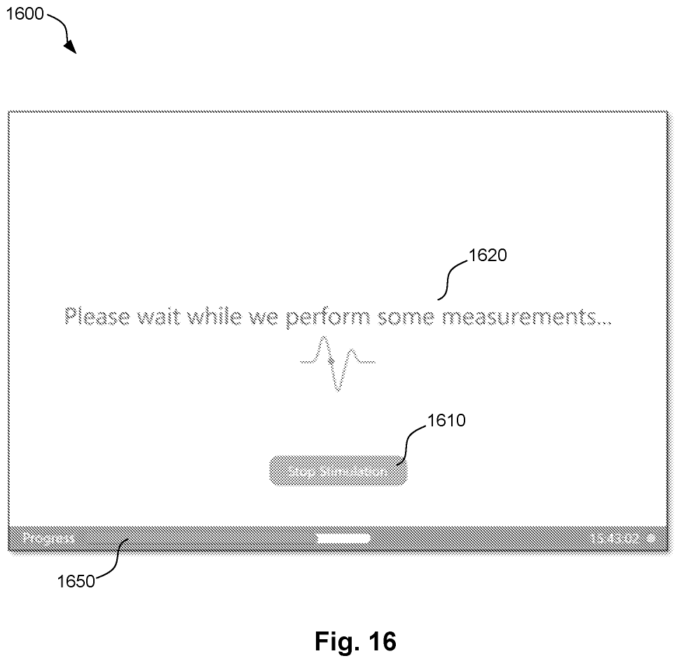

In one implementation of the MO stage 830, the APM displays on the UI display of the CI 740 a screen 1600 as illustrated in FIG. 16. The screen 1600 comprises some information 1620, a progress bar 1650, and a Stop Stimulation control 1610. The screen 1600 is displayed while some neural stimulation is delivered, and the collected signal windows are analysed as described below. In one implementation, activation of the Stop Stimulation control 1610 at any time during the MO stage stops the stimulation. The screen 1600 is then replaced with an exit screen (not shown) informing the patient that manual programming is required. The MO stage 830 ends and the APM then halts without loading a program to the device 710.

The progress bar 1650, like the progress bars 1050, 1250, and 1550, indicates approximate quantitative progress through the entire workflow 800.

Once the data collection and analysis of the MO stage 830 are complete, the APM displays one of two screens depending on the success of the data analysis. If the data analysis was successful, the screen contains a Finish control. Instructions on the screen inform the patient that the programming was successful. When the patient activates the Finish control, the MO stage 830 ends.

If the data analysis was unsuccessful, the screen contains a Finish control. Instructions on the screen inform the patient that the programming was unsuccessful, and that manual programming is required. When the patient activates the Finish control, the MO stage 830 ends and the APM halts without loading a program to the device 710.

### Data Analysis During the MO Stage

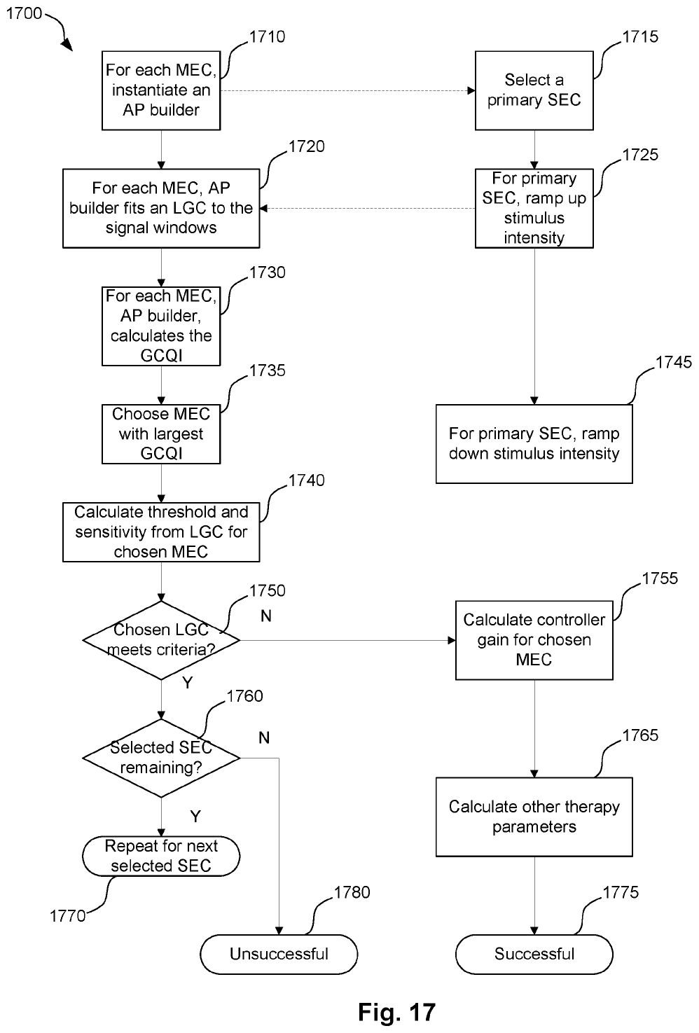

FIG. 17 contains a flowchart illustrating a data collection and analysis method 1700 carried out by the APM and the device 710 during the MO stage 830 according to one implementation of the APM. The method 1700 starts at step 1715, where the APM selects a primary SEC from among the selected SECs from the coverage selection stage 820. In one implementation, step 1715 selects as the primary SEC the remaining selected SEC (if there is one) with the smallest comfortable stimulus intensity. Meanwhile, step 1710 instantiates an AP builder for each MEC corresponding to the current primary SEC, as in step 1110. The MECs corresponding to one SEC are illustrated in FIG. 9. At the next step 1725, the APM instructs the device 710 to commence a stimulus ramp for the current primary SEC. The stimulus ramp commences at a stimulus intensity of zero and increases via discrete steps to a maximum stimulus intensity determined by the Max value for the primary SEC selected at step 1715. In one implementation, there are 10 evenly-spaced steps to a maximum stimulus intensity that is 90% of the Max value for the primary SEC.

In an alternative implementation of step 1725, the device 710 may increase the stimulus intensity in constant-ratio steps, i.e. each increment comprises multiplying the previous stimulus current amplitude by a constant ratio. This is equivalent to a ramp with an exponential rather than a linear profile. In an exponential ramp, the discrete steps are more widely spaced as the maximum stimulus intensity is approached. In one example, if the ECAP threshold is set to 0.7 times the Max value as described above, a constant ratio of 1.025 will provide ten steps of exponential increase between the ECAP threshold and 90% of Max.

In an alternative implementation of step 1725, rather than using a ramp, the device 710 may vary the stimulus intensity non-monotonically between the zero and the maximum stimulus intensity. For example, the variation may be random. Such an approach may lead to faster convergence by the AP builder to the fitted LGC.

During the stimulus ramp, at step 1720, the APM instructs the device 710 to capture and return signal windows for each stimulus current amplitude s at each MEC. The returned signal windows for each MEC are analysed by the corresponding AP builder at step 1720, which extracts a detected ECAP amplitude d from each signal window. In one implementation, multiple signal windows (e.g. 16) are analysed for each MEC at each stimulus current amplitude s during the ramp. Each AP builder tunes the parameters, e.g. length and correlation delay, of its ECAP detector during the step 1720 using the captured signal windows as described above.

To complete step 1720, each AP builder fits an LGC to the set of (s, d) value pairs for the corresponding MEC as described above. Meanwhile, at step 1745, the APM instructs the device 710 to ramp down the stimulus intensity. In one implementation, step 1745 uses a threshold ramp as described above, using the ECAP threshold as the threshold of the threshold ramp.

Each AP builder then at step 1730 calculates the GCQI of the LGC fit for the corresponding MEC as described above. At step 1735, the APM chooses the MEC corresponding to the fitted LGC with the highest GCQI. The APM then at step 1740 calculates the ECAP threshold and patient sensitivity S from the fitted LGC as described above.

Step 1750 then determines whether the chosen MEC meets certain exclusion criteria indicative of poor quality. In one implementation, the exclusion criteria are:
The chosen GCQI is less than a threshold, e.g. 10 dB.
The calculated ECAP threshold is outside a predetermined range. In one implementation, the range is from the first percentile to the 99th percentile of the distribution of ECAP thresholds obtained from existing patient data.
The calculated sensitivity is outside a predetermined range. In one implementation, the range is from the first percentile to the 99th percentile of the distribution of patient sensitivities obtained from existing patient data.

If any of the exclusion criteria are met (“Y”), the current primary SEC is marked as unsuccessful. The APM at step 1760 determines whether there are any remaining selected SECs that have not been tested. If so (“Y”), step 1770 restarts the method 1700. If not (“N”), the final step 1780 ends the MO stage 830, and the workflow 800 is deemed unsuccessful.

If none of the exclusion criteria tested at step 1750 are met (“N”), the current primary SEC is marked as be the primary SEC for the program, and the chosen MEC is marked as the optimal MEC for the primary SEC. Step 1755 then calculates the gain K of the gain element 336 of the system 300 from the patient sensitivity S calculated at step 1740. In one implementation, step 1755 calculates the gain K as

$$K = \frac{cos ω - 1 + \sqrt{{cos}^{2} ω - 4 cos ω + 3}}{S} \qquad ( 11 )$$

where

$$ω = \frac{2 π {f}_{c}}{{f}_{s}} ,$$

f_{c} is a loop cutoff frequency, and fₛ is the stimulus frequency. In one implementation, the loop cutoff frequency is set to 3 Hz to balance the attenuation of noise with the attenuation of postural disturbances such as heartbeat.

Step 1765 calculates other therapy parameters for the CLNS system 300. In one implementation, the therapy parameters are:
A target ECAP amplitude. This may be calculated using equation (7) as the value of ECAP amplitude d on the fitted LGC corresponding to the comfortable stimulus intensity s=I_{comf}.
A maximum stimulus intensity. This may be set to the Max value for the primary SEC.
A maximum target ECAP amplitude. This may be set to the value of ECAP amplitude d on the fitted LGC corresponding to the Max value for the primary SEC.

Step 1775 saves the determined program, comprising the selected SECs, including the primary SEC, the optimal MEC, the Max, ECAP threshold, and sensitivity, and calculated therapy parameters. The MO stage 830 ends, and the workflow 800 is deemed successful.

In an alternative implementation of the MO stage 830, there is no primary SEC. Instead, each selected SEC runs its own independent feedback loop via its own dedicated MEC, assuming an MEC of sufficient quality may be found. A modified method 1700 is carried out for each selected SEC. The modified method 1700 has no step 1715, nor does it have steps 1760 and 1770. Instead, if one of the exclusion criteria is met at step 1750, the modified method 1700 ends unsuccessfully at step 1780.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not limiting or restrictive.

LABEL LIST

| | |
|---|---|
| implanted stimulator | 100 |
| patient | 108 |
| electronics module | 110 |
| battery | 112 |
| telemetry module | 114 |
| controller | 116 |
| memory | 118 |
| clinical data | 120 |
| clinical settings | 121 |
| control programs | 122 |
| pulse generator | 124 |
| electrode selection module | 126 |
| measurement circuit | 128 |
| system ground | 130 |
| electrode array | 150 |
| current pulse | 160 |
| neural response | 170 |
| nerve | 180 |
| communications channel | 190 |
| external computing device | 192 |
| CLNS system | 300 |
| clinical settings controller | 302 |
| target ECAP controller | 304 |
| box | 308 |
| box | 309 |
| feedback controller | 310 |
| box | 311 |
| stimulator | 312 |
| element | 313 |
| measurement circuitry | 318 |
| ECAP detector | 320 |
| comparator | 324 |
| gain element | 336 |
| integrator | 338 |
| activation plot | 402 |
| ECAP threshold | 404 |
| discomfort threshold | 408 |
| perception threshold | 410 |
| therapeutic range | 412 |
| activation plot | 502 |
| activation plot | 504 |
| activation plot | 506 |
| ECAP threshold | 508 |
| ECAP threshold | 510 |
| ECAP threshold | 512 |
| ECAP target | 520 |
| ECAP | 600 |
| neural stimulation system | 700 |
| device | 710 |
| remote controller | 720 |
| CST | 730 |
| CI | 740 |
| charger | 750 |
| workflow | 800 |
| PCSR stage | 810 |
| coverage survey stage | 815 |
| coverage selection stage | 820 |
| MO stage | 830 |
| table | 900 |
| graphical representation | 910 |
| screen | 1000 |
| stimulation control | 1010 |
| instructions | 1020 |
| next control | 1040 |
| progress bar | 1050 |
| sector | 1060 |
| method | 1100 |
| method | 1100a |
| step | 1110 |
| step | 1115 |
| step | 1120 |
| step | 1125 |
| step | 1130 |
| step | 1130a |
| step | 1135 |
| step | 1140 |
| step | 1145 |
| step | 1145a |
| step | 1150 |
| step | 1155 |
| step | 1155a |
| step | 1160 |
| step | 1160a |
| step | 1163 |
| step | 1165 |
| step | 1168 |
| step | 1170 |
| step | 1175 |
| step | 1180 |
| screen | 1200 |
| stimulation control | 1210 |
| instructions | 1220 |
| options | 1230 |
| next control | 1240 |
| progress bar | 1250 |
| LGC model | 1310 |
| linear model | 1320 |
| arrow | 1330 |
| profile | 1400 |
| target current amplitude | 1410 |
| profile | 1420 |
| instant | 1430 |
| interval | 1440 |
| interval | 1450 |
| threshold current amplitude | 1460 |
| screen | 1500 |
| toggle tile | 1510a |
| toggle tile | 1510b |
| toggle tile | 1510c |
| toggle switch | 1520b |
| toggle switch | 1520c |
| instructions | 1530 |
| next control | 1540 |
| progress bar | 1550 |
| control | 1560 |
| screen | 1600 |
| stimulation control | 1610 |
| information | 1620 |
| progress bar | 1650 |
| method | 1700 |
| step | 1710 |
| step | 1715 |
| step | 1720 |
| step | 1725 |
| step | 1730 |
| step | 1735 |
| step | 1740 |
| step | 1745 |
| step | 1750 |
| step | 1755 |
| step | 1760 |
| step | 1765 |
| step | 1770 |
| step | 1775 |
| step | 1780 |
| ramp | 1800 |
| star | 1805 |
| communication | 1810 |
| cross | 1815 |
| ramp | 1820 |
| communication | 1825 |
| first timeout period | 1830 |
| second timeout period | 1835 |
| communication | 1837 |
| ramp | 1840 |
| communication | 1845 |
| ramp | 1850 |
| first timeout period | 1855 |
| second timeout period | 1860 |
| down - ramp | 1870 |
| down - ramp command | 1875 |
| communication | 1880 |
| down - ramp | 1890 |

1. A neurostimulation system comprising:
a neurostimulation device for controllably delivering neural stimuli, the neurostimulation device comprising:
a stimulus source configured to deliver neural stimuli via selected electrodes of a plurality of implantable electrodes to a neural pathway of a patient; and
a control unit configured to control the stimulus source to deliver each neural stimulus according to a stimulus intensity parameter; and
an external computing device in communication with the neurostimulation device, the external computing device comprising:
a display, and
a processor configured to:
initialise the stimulus intensity parameter;
render a stimulation control on the display;
ramp, on receiving an activation of the stimulation control by a user, a value of the stimulus intensity parameter while instructing the control unit to control the stimulus source to deliver the neural stimuli according to the ramping value of the stimulus intensity parameter; and
cease, upon receiving a de-activation of the stimulation control,
ramping the value of the stimulus intensity parameter.

2. The neurostimulation system of claim 1, wherein the processor is further configured to ramp down, upon receiving the de-activation, the value of the stimulus intensity parameter, while instructing the control unit to control the stimulus source to deliver the neural stimuli according to the down-ramping value of the stimulus intensity parameter.

3. The neurostimulation system of claim 1, wherein the processor is further configured to instruct, upon receiving the de-activation, the stimulus source to cease delivering the neural stimuli.

4. The neurostimulation system of claim 1, wherein the processor is further configured to record, upon receiving the de-activation, the value of the stimulus intensity parameter as a discomfort threshold for the selected electrodes.

5. The neurostimulation system of claim 4, wherein the processor is further configured to program, using the recorded value of the stimulus intensity parameter, the neurostimulation device to deliver neural stimuli to the patient.

6. The neurostimulation system of claim 1, wherein the stimulation control is configured to:
become activated upon the user interacting with the stimulation control; and
remain activated as long as the user continues to interact with the stimulation control; and
become de-activated as soon as the user ceases to interact with the stimulation control.

7. The neurostimulation system of claim 1, wherein the processor is configured to animate the stimulation control to indicate elapsed time since the activation of the control.

8. The neurostimulation system of claim 7, wherein the processor is configured to animate the stimulation control to indicate that the stimulus intensity has reached a neural response threshold.

9. The neurostimulation system of claim 1, wherein the processor is further configured to cease ramping the value of the stimulus intensity parameter upon expiry of a first timeout period since receipt of a communication from the neurostimulation device.

10. The neurostimulation system of claim 9, wherein the processor is further configured to ramp down, upon expiry of a second timeout period since the expiry of the first timeout period without receiving a communication from the neurostimulation device, the value of the stimulus intensity parameter, while instructing the control unit to control the stimulus source to deliver the neural stimuli according to the down-ramping value of the stimulus intensity parameter.

11. An automated method of controlling a neurostimulation device to deliver neural stimuli using an external computing device in communication with the neurostimulation device, the method comprising:
rendering, by a processor of the external computing device, a stimulation control on a display of the external computing device;
ramping, by the processor, on receiving an activation of the stimulation control by a user, a value of a stimulus intensity parameter, while instructing the neurostimulation device to deliver the neural stimuli according to the ramping value of the stimulus intensity parameter via selected electrodes of a plurality of implanted electrodes; and
ceasing, by the processor, upon receiving a de-activation of the stimulation control, to ramp the value of the stimulus intensity parameter.

12. The method of claim 11, further comprising ramping down, by the processor, upon receiving the de-activation, the value of the stimulus intensity parameter, while instructing the neurostimulation device to deliver the neural stimuli according to the down-ramping value of the stimulus intensity parameter via the selected electrodes.

13. The method of claim 11, further comprising ramping down, by the processor, upon the stimulus intensity parameter satisfying a predetermined condition, the value of the stimulus intensity parameter to zero, while instructing the neurostimulation device to deliver the neural stimuli according to the down-ramping value of the stimulus intensity parameter via the selected electrodes.

14. The method of claim 13, wherein the predetermined condition comprises the stimulus intensity parameter reaching a hard ceiling.

15. The method of claim 12, wherein the down-ramping value of the stimulus intensity parameter follows a linear profile with a predetermined rate of decrease.

16. The method of claim 15, wherein a rate of the linear profile is chosen such that the stimulus intensity parameter reaches zero after a predetermined interval.

17. The method of claim 12, wherein the down-ramping value of the stimulus intensity parameter follows a threshold ramp parametrised by an ECAP threshold.

18. The method of claim 11, wherein the ramp is linear with time.

19. The method of claim 18, wherein a ramp rate of the linear ramp is predetermined.

20. The method of claim 11, wherein the ramp is a threshold ramp parametrised by an ECAP threshold.

21. The method of claim 11, further comprising instructing, by the processor, upon receiving the de-activation, the neurostimulation device to cease delivering the neural stimuli.

22. The method of claim 11, further comprising recording, upon receiving the de-activation, the value of the stimulus intensity parameter as a discomfort threshold for the selected electrodes of the plurality of implanted electrodes.

23. The method of claim 11, further comprising repeating, by the processor, the ramping and instructing and ceasing for further selected electrodes of the plurality of implanted electrodes.

24. The method of claim 11, wherein the stimulus intensity parameter is an amplitude of a stimulus current pulse.

25. The method of claim 11, further comprising animating the stimulation control to indicate elapsed time since the activation of the control.

26. The method of claim 25, wherein the animating indicates that the stimulus intensity has reached a neural response threshold.

27. The method of claim 11, further comprising ceasing ramping the value of the stimulus intensity parameter upon expiry of a first timeout period since receipt of a communication from the neurostimulation device.

28. The method of claim 27, further comprising ramping down, upon expiry of a second timeout period since the expiry of the first timeout period without receiving a communication from the neurostimulation device, the value of the stimulus intensity parameter, while instructing, by the processor, the neurostimulation device to deliver the neural stimuli according to the down-ramping value of the stimulus intensity parameter.
